(19) United States
(12) Patent Application Publication
(10) Pub. No.: US 2025/0188130 A1
(43) Pub. Date: Jun. 12, 2025

(54) COMPOSITIONS COMPRISING MODIFIED FLAVIVIRUS E GLYCOPROTEINS AND METHODS OF MAKING AND USING THE SAME

(71) Applicant: The University of North Carolina at Chapel Hill, Chapel Hill, NC (US)

(72) Inventors: Ralph Baric, Haw River, NC (US); Long Ping Victor Tse, Kirkwood, MO (US); Rita Meganck, Kirkwood, MO (US)

(21) Appl. No.: 18/846,485

(22) PCT Filed: Mar. 17, 2023
(86) PCT No.: PCT/US2023/064632

§ 371 Date: Sep. 12, 2024

Related U.S. Application Data
(60) Provisional application No. 63/320,922, filed on Mar. 17, 2022.

(51) Int. Cl. C07K 14/005; A61K 39/12; A61P 37/04

(52) U.S. Cl. CPC C07K 14/005; A61K 39/12; A61P 37/04; C07K 2319/95; C12N 2770/24122; C12N 2770/24123; C12N 2770/24134; C12N 2770/24151

(57) ABSTRACT

This invention relates to mature flavivirus particles and methods of making and using the same. This invention further relates to flavivirus E glycoproteins, nucleic acids encoding the same, as well as particles, populations, and compositions comprising the same. Also disclosed are methods of making and using the E glycoproteins of the invention.

### STATEMENT OF PRIORITY

This application claims the benefit, under 35 U.S.C. § 119(e), of U.S. Provisional Application No. 63/320,922, filed Mar. 17, 2022, the entire contents of which is incorporated by reference herein in its entirety.

### STATEMENT OF GOVERNMENT SUPPORT

This invention was made with government support under Grant Numbers AI107731, AI106695 and AI125198 awarded by the National Institutes of Health. The government has certain rights in the invention.

### STATEMENT REGARDING ELECTRONIC FILING OF A SEQUENCE LISTING

A Sequence Listing in XML format, entitled 5470-928WO_ST26.xml, 27,861 bytes in size, generated on Mar. 17, 2023, and filed herewith, is hereby incorporated by reference in its entirety for its disclosures.

### FIELD OF THE INVENTION

This invention is directed to methods and compositions for flavivirus therapeutics, such as vaccines, and diagnostics.

### BACKGROUND OF THE INVENTION

Dengue virus (DENV) is a member of the Flavivirus genus and is a major global public health threat. Dengue affects ˜400 million people each year, of which ˜20% will develop severe Dengue Hemorrhagic Fever/Dengue Shock Syndrome (DHF/DSS) (Bhatt et al. 2013 Nature 496(7446):504-507; Brady et al. 2016 Environ Res 151:115-123). DENV is transmitted through Aedes mosquito vectors, and global warming is increasing the endemic range of Dengue worldwide. The etiology of Dengue is unique, as first-time infections are rarely severe. However, re-infection with a different serotype (four major serotypes of DENV are found worldwide) dramatically increases the risk of DHF/DSS (Halstead, S. B. 1988 Science 239:476-481). This is due to the phenomenon of antibody dependent enhancement (ADE), in which sub-neutralizing concentrations of antibodies (Abs) targeting DENV lead to enhanced viral uptake and infection in an Fc-receptor mediated manner (Katzelnick et al. 2017 Science 358:929-932).

The present invention overcomes previous shortcomings in the art by providing recombinant flavivirus E glycoproteins with modified fusion loop epitopes and reduced ability to stimulate targeting and/or neutralization by ADE-associated anti-flavivirus antibodies.

### SUMMARY OF THE INVENTION

One aspect of the present invention provides a recombinant flavivirus E glycoprotein comprising a flavivirus E glycoprotein backbone comprising a fusion loop epitope, and one or more of the following amino acid substitutions in the fusion loop epitope, wherein the numbering corresponds to the amino acid sequence of SEQ ID NO:1[wt DV2 E]: 103S and/or 106L.

In some embodiments, the recombinant flavivirus E glycoprotein of the present invention may further comprise one or more amino acid substitution(s) in position 101, 102, 104, 105, and/or 107.

Also provided are isolated nucleic acid molecules (e.g., mRNA molecules such as mRNA-based prophylactic and/or therapeutic vaccine molecules), flavivirus particles or virus like particles (VLP), vectors, populations of flavivirus particles, compositions, and pharmaceutical compositions (e.g., pharmaceutical formulations) comprising the recombinant flavivirus E glycoprotein of the present invention.

In some embodiments, aspects of the invention may further comprise and/or encode a modified flavivirus prM glycoprotein.

In some embodiments, particles of the invention (e.g., flavivirus particles or VLP) may be in a mature form.

Another aspect of the invention provides a method of producing an immune response to a flavivirus in a subject, comprising administering to the subject an effective amount of the E glycoprotein, flavivirus particle or VLP, nucleic acid molecule, vector, population, and/or composition of the present invention.

Another aspect of the invention provides a method of treating a flavivirus infection in a subject, comprising administering to the subject an effective amount of the E glycoprotein, flavivirus particle or VLP, nucleic acid molecule, vector, population, and/or composition of the present invention.

Another aspect of the invention provides a method of preventing a disorder associated with a flavivirus infection in a subject, comprising administering to the subject an effective amount of the E glycoprotein, flavivirus particle or VLP, nucleic acid molecule, vector, population, and/or composition of the present invention.

Another aspect of the invention provides a method of protecting a subject from the effects of a flavivirus infection, comprising administering to the subject an effective amount of the E glycoprotein, flavivirus particle or VLP, nucleic acid molecule, vector, population, and/or composition of the present invention.

Another aspect of the invention provides a method of identifying the presence of a neutralizing antibody to a flavivirus in a biological sample from a subject, comprising: a) administering a composition comprising the E glycoprotein of the invention to the subject in an amount effective to induce an antibody response to the E glycoprotein; b) contacting a biological sample from the subject with flavivirus particles comprising the E glycoprotein of step (a) above under conditions whereby neutralization of the flavivirus particles can be detected; and c) detecting neutralization in step (b), thereby identifying the presence of a neutralizing antibody to the flavivirus in the biological sample from the subject.

Another aspect of the invention provides a method of identifying the presence of a neutralizing antibody to a flavivirus in a biological sample from a subject, comprising: a) contacting a biological sample from a subject that has been administered a E glycoprotein of the invention with flavivirus particles comprising the E glycoprotein under conditions whereby neutralization of the flavivirus particles can be detected; and b) detecting neutralization in step (a), thereby identifying the presence of a neutralizing antibody to the flavivirus in the biological sample from the subject.

Another aspect of the invention provides a method of identifying an immunogenic composition that induces a neutralizing antibody to a flavivirus in a subject, the method comprising: a) administering an immunogenic composition comprising the E glycoprotein of the invention to a subject in an amount effective to induce an antibody response to the E glycoprotein; b) contacting a biological sample from the subject with flavivirus particles comprising the E glycoprotein of step (a) under conditions whereby neutralization of the flavivirus particles can be detected; c) determining if the biological sample comprises an antibody that neutralizes flavivirus particles comprising the E glycoprotein of step (a); and d) identifying the immunogenic composition as inducing a neutralizing antibody to the flavivirus in the subject if the biological sample comprises an antibody that neutralizes flavivirus particles comprising the E glycoprotein of (a).

Another aspect of the invention provides a method of identifying an immunogenic composition that induces a neutralizing antibody to a flavivirus in a subject, the method comprising: a) contacting a biological sample from a subject that has been administered an immunogenic composition comprising the E glycoprotein of the invention with flavivirus particles comprising the E glycoprotein under conditions whereby neutralization of the flavivirus particles can be detected; b) determining if the biological sample comprises an antibody that neutralizes flavivirus particles comprising the E glycoprotein of step (a); and c) identifying the immunogenic composition as inducing a neutralizing antibody to the flavivirus in the subject if the biological sample comprises an antibody that neutralizes flavivirus particles comprising the E glycoprotein of (a).

Another aspect of the invention provides a method of detecting an antibody to a flavivirus in a sample, comprising: a) contacting the sample with the E glycoprotein of any of the invention under conditions whereby an antigen/antibody complex can form; and b) detecting formation of an antigen/antibody complex, thereby detecting an antibody to the flavivirus in the sample.

Another aspect of the invention provides a method of identifying an antibody to a flavivirus in a biological sample from a subject, comprising: a) administering a composition comprising the E glycoprotein of the invention to the subject in an amount effective to induce an antibody response to the E glycoprotein; b) contacting a biological sample from the subject with the E glycoprotein of (a) under conditions whereby an antigen/antibody complex can form; and c) detecting formation of an antigen/antibody complex, thereby identifying an antibody to the flavivirus in the biological sample from the subject.

Another aspect of the invention provides method of identifying an antibody to a flavivirus in a biological sample from a subject, comprising: a) contacting a biological sample from a subject that has been administered an immunogenic composition comprising the E glycoprotein of the invention with the E glycoprotein under conditions whereby an antigen/antibody complex can form; and b) detecting formation of an antigen/antibody complex, thereby identifying an antibody to the flavivirus in the biological sample from the subject.

Another aspect of the invention provides a method of identifying an immunogenic composition that induces an antibody to a flavivirus in a subject, the method comprising: a) contacting a biological sample from a subject that has been administered an immunogenic composition comprising an E glycoprotein of the invention with the E glycoprotein under conditions whereby an antigen/antibody complex can form; and b) detecting formation of an antigen/antibody complex, thereby identifying an immunogenic composition that induces an antibody to the flavivirus in the subject.

Another aspect of the invention provides a method of identifying an immunogenic composition that induces a neutralizing antibody to a flavivirus in a subject, comprising: a administering an immunogenic composition comprising an E glycoprotein of the invention to a subject in an amount effective to induce an antibody response to the E glycoprotein; b) contacting a biological sample from the subject with the E glycoprotein of (a) under conditions whereby an antigen/antibody complex can form; and c) detecting formation an antigen/antibody complex, thereby identifying an immunogenic composition that induces a neutralizing antibody to the flavivirus in the subject.

Another aspect of the invention provides a method of producing a flavivirus particle, comprising: constructing a flavivirus particle comprising the recombinant E glycoprotein of the invention; thereby producing a flavivirus particle comprising the recombinant E glycoprotein.

### BRIEF DESCRIPTION OF THE DRAWINGS

FIG. 1 shows schematic diagrams related to the generation of DENV2 fusion loop mutants via directed evolution. FIG. 1 panel A) Alignment of Top: Dengue virus fusion loops; Bottom: Mosquito-borne virus fusion loops, including Yellow Fever virus (YFV), Zika virus (ZIKV), West Nile virus (WNV), Kunjin virus (KUNV), Murray Valley Encephalitis virus (MVEV), Japanese Encephalitis virus (JEV), Usuthu virus (USUV), and Saint Louis Encephalitis virus (SLEV) Sequence shown is SEQ ID NO:18. FIG. 1 panel B) Schematic of directed evolution procedure. Saturation mutagenesis libraries were used to produce virus, which was passaged three times in either C6/36 or Vero 81 cells. At the end of selection, viral genomes were isolated and mutations identified by high-throughput sequencing. Library consensus sequences are SEQ ID NO:13 and SEQ ID NO:14. FIG. 1 panel C) Left: Bubble plot of the sequences identified from either the unselected or selected (passage 3) C6/36 DENV libraries, with highlighted SEQ ID NO:10. Right: Pie chart of the sequences from passage 3 C6/36 DENV libraries, with highlighted SEQ ID NO:10. FIG. 1 panel D) Structure of the DENV envelope with the fusion loop mutations highlighted in red. FIG. 1 panel E) Sequences of the fusion loop and furin cleavage site, respectively, of DENV2 (SEQ ID NO:18; SEQ ID NO:19), DENV2-FL (SEQ ID NO: 10; SEQ ID NO:19), and DENV2-FL-Mature (SEQ ID NO:10; SEQ ID NO:11).

FIG. 2 shows data plots related to physical and biological properties of mature DENV2 fusion loop mutants. FIG. 2 panel A) Growth curves of DENV2-WT, DENV2-FL, and DENV-FL-Mature on C6/36 cells (left) or Vero 81 cells (right). FIG. 2 panel B) Thermostability assay on DENV2-WT, DENV2-FL, and DENV2-FL-Mature. FIG. 2 panel C) Western blot of virions, blotted against Envelope and prM proteins. The prM:E ratio was determined and normalized to the DENV2-WT ratio. Averages of 3 biological replicates are shown. Student's t-test is used for statistical comparison: ns=not significant; *<0.05; **<0.005; ***<0.0005.

FIG. 3 shows data plots related to Fusion loop mutant insensitivity to fusion loop monoclonal Abs, the major target for cross-reactive Abs in NHPs. FIG. 3 panel A) EC50 values for neutralization of DENV2-WT and DENV2-FL-Mature with mAbs against DENV2 prM (2H2, 1E16, 5M22), EDI/II (3F9), EDE (C10, B7), EDIII (2D22), or fusion loop (1M7, 1N5, 1L6, 4G2). FIG. 3 panel B) Neutralization curves for neutralization of DENV2-WT and DENV2-FL-Mature with mAbs against the DENV2 fusion loop. FIG. 3 panel C) Neutralization of DENV2-WT and DENV2-FL-Mature with sera from NHPs vaccinated with either DENV4 or DENV2. FIG. 3 panel D) Additional data plots from experiments as described and shown in FIGS. 3B and 3C. The nucleic and amino acid sequences of the shown “VT151” and “VT163” (DV2-FL-Mature) are provided in the SEQUENCES section as SEQ ID NOs:15-17.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, +5%, +1%, +0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the term “nucleic acid” encompasses both RNA and DNA, including cDNA, genomic DNA, synthetic (e.g., chemically synthesized) DNA and chimeras of RNA and DNA. The nucleic acid may be double-stranded or single-stranded. The nucleic acid may be synthesized using nucleotide analogs or derivatives (e.g., inosine or phosphorothioate nucleotides). Such nucleotides can be used, for example, to prepare nucleic acids that have altered base-pairing abilities or increased resistance to nucleases.

The terms “nucleic acid segment,” “nucleotide sequence,” “nucleic acid molecule,” or more generally “segment” will be understood by those in the art as a functional term that includes both genomic DNA sequences, ribosomal RNA sequences, transfer RNA sequences, messenger RNA sequences, small regulatory RNAs, operon sequences and smaller engineered nucleotide sequences that express or may be adapted to express, proteins, polypeptides or peptides. Nucleic acids of the present disclosure may also be synthesized, either completely or in part, by methods known in the art. Thus, all or a portion of the nucleic acids of the present codons may be synthesized using codons preferred by a selected host. Species-preferred codons may be determined, for example, from the codons used most frequently in the proteins expressed in a particular host species. Other modifications of the nucleotide sequences may result in mutants having slightly altered activity.

The term “sequence identity,” as used herein, has the standard meaning in the art. As is known in the art, a number of different programs can be used to identify whether a polynucleotide or polypeptide has sequence identity or similarity to a known sequence. Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J. Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABIOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol. 266:460 (1996); blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

As used herein, the term “polypeptide” encompasses both peptides and proteins (including fusion proteins), unless indicated otherwise.

As used herein, the term “chimera,” “chimeric,” and/or “fusion protein” refer to an amino acid sequence (e.g., polypeptide) generated non-naturally by deliberate human design comprising, among other components, an amino acid sequence of a protein of interest and/or a modified variant and/or active fragment thereof (a “backbone”), wherein the protein of interest comprises modifications (e.g., substitutions such as singular residues and/or contiguous regions of amino acid residues) from different wild type reference sequences (chimera), optionally linked to other amino acid segments (fusion protein). The different components of the designed protein may provide differing and/or combinatorial function. Structural and functional components of the designed protein may be incorporated from differing and/or a plurality of source material. The designed protein may be delivered exogenously to a subject, wherein it would be exogenous in comparison to a corresponding endogenous protein.

As used herein with respect to nucleic acids, the term “operably linked” refers to a functional linkage between two or more nucleic acids. For example, a promoter sequence may be described as being “operably linked” to a heterologous nucleic acid sequence because the promoter sequence initiates and/or mediates transcription of the heterologous nucleic acid sequence. In some embodiments, the operably linked nucleic acid sequences are contiguous and/or are in the same reading frame.

In embodiments of the invention, an “immunogenically active fragment” of a flavivirus polypeptide (e.g., the E protein) comprises, consists essentially of or consists of at least about 6, 8, 10, 12, 15, 20, 30, 50, 75, 100, 125, 150, 200, 250, 300, 350, 400, 450 or more amino acids, optionally contiguous amino acids, and/or less than about 495, 475, 450, 425, 400, 350, 300, 250, 200, 150, 100, 75 or 50 amino acids, optionally contiguous amino acids, including any combination of the foregoing as long as the lower limit is less than the upper limit, and the “immunogenically active fragment” induces an immune response (e.g., IgG and/or IgA that react with the native antigen), optionally a protective immune response, against dengue virus in a host and induces the production of antibodies that specifically bind to the quaternary dengue virus epitope newly identified by the inventors.

The term “epitope” as used herein means a specific amino acid sequence that, when present in the proper conformation, provides a reactive site for an antibody (e.g., B cell epitope) or T cell receptor (e.g., T cell epitope).

Portions of a given polypeptide that include a B-cell epitope can be identified using any number of epitope mapping techniques that are known in the art. (See, e.g., Epitope Mapping Protocols in Methods in Molecular Biology, Vol. 66, Glenn E. Morris, Ed., 1996, Humana Press, Totowa, N.J.). For example, linear epitopes can be determined by, e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715.

Similarly, conformational epitopes can be readily identified by determining spatial conformation of amino acids such as by, e.g., x-ray crystallography and 2-dimensional nuclear magnetic resonance. Antigenic regions of proteins can also be identified using standard antigenicity and hydropathy plots, such as those calculated using, e.g., the Omiga version 1.0 software program available from the Oxford Molecular Group. This computer program employs the Hopp/Woods method (Hopp et al. Proc. Natl. Acad. Sci USA (1981) 78:3824-3828) for determining antigenicity profiles and the Kyte-Doolittle technique (Kyte et al. J. Mol. Biol. (1982) 157:105-132) for hydropathy plots.

Generally, T-cell epitopes that are involved in stimulating the cellular arm of a subject's immune system are short peptides of about 8-25 amino acids. A common way to identify T-cell epitopes is to use overlapping synthetic peptides and analyze pools of these peptides, or the individual ones, that are recognized by T cells from animals that are immune to the antigen of interest, using, for example, an enzyme-linked immunospot assay (ELISPOT). These overlapping peptides can also be used in other assays such as the stimulation of cytokine release or secretion, or evaluated by constructing major histocompatibility (MHC) tetramers containing the peptide. Such immunogenically active fragments can also be identified based on their ability to stimulate lymphocyte proliferation in response to stimulation by various fragments from the antigen of interest.

A “recombinant” nucleic acid, polynucleotide or nucleotide sequence is one produced by genetic engineering techniques.

A “recombinant” polypeptide is produced from a recombinant nucleic acid, polypeptide or nucleotide sequence.

As used herein, an “isolated” polynucleotide (e.g., an “isolated nucleic acid” or an “isolated nucleotide sequence”) means a polynucleotide at least partially separated from at least some of the other components of the naturally occurring organism or virus, for example, the cell or viral structural components or other polypeptides or nucleic acids commonly found associated with the polynucleotide. Optionally, but not necessarily, the “isolated” polynucleotide is present at a greater concentration (i.e., is enriched) as compared with the starting material (e.g., at least about a two-fold, three-fold, four-fold, ten-fold, twenty-fold, fifty-fold, one-hundred-fold, five-hundred-fold, one thousand-fold, ten thousand-fold or greater concentration). In representative embodiments, the isolated polynucleotide is at least about 1%, 5%, 10%, 20%, 30%, 40%, 50%, 60%, 70%, 80%, 90%, 95% or more pure.

An “isolated” polypeptide means a polypeptide that is at least partially separated from at least some of the other components of the naturally occurring organism or virus, for example, the cell or viral structural components or other polypeptides or nucleic acids commonly found associated with the polypeptide. Optionally, but not necessarily, the “isolated” polypeptide is present at a greater concentration (i.e., is enriched) as compared with the starting material (e.g., at least about a two-fold, three-fold, four-fold, ten-fold, twenty-fold, fifty-fold, one-hundred-fold, five-hundred-fold, one thousand-fold, ten thousand-fold or greater concentration). In representative embodiments, the isolated polypeptide is at least about 1%, 5%, 10%, 20%, 30%, 40%, 50%, 60%, 70%, 80%, 90%, 95% or more pure.

Furthermore, an “isolated” cell is a cell that has been partially or completely separated from other components with which it is normally associated in nature. For example, an isolated cell can be a cell in culture medium and/or a cell in a pharmaceutically acceptable carrier.

The term “endogenous” refers to a component naturally found in an environment, i.e., a gene, nucleic acid, miRNA, protein, cell, or other natural component expressed in the subject, as distinguished from an introduced component, i.e., an “exogenous” component.

As used herein, the term “heterologous” refers to a nucleotide/polypeptide that originates from a foreign species, or, if from the same species, is substantially modified from its native form in composition and/or genomic locus by deliberate human intervention.

As used herein with respect to nucleic acids, the term “fragment” refers to a nucleic acid that is reduced in length relative to a reference nucleic acid and that comprises, consists essentially of and/or consists of a nucleotide sequence of contiguous nucleotides identical or almost identical (e.g., 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. In some embodiments, the nucleic acid fragment comprises, consists essentially of or consists of at least about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive nucleotides. In some embodiments, the nucleic acid fragment comprises, consists essentially of or consists of less than about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive nucleotides.

As used herein with respect to polypeptides, the term “fragment” refers to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive amino acids. In some embodiments, the polypeptide fragment comprises, consists essentially of or consists of less than about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive amino acids.

As used herein with respect to nucleic acids, the term “functional fragment” or “active fragment” refers to nucleic acid that encodes a functional fragment of a polypeptide.

As used herein with respect to polypeptides, the term “functional fragment” or “active fragment” refers to polypeptide fragment that retains at least about 20%, 25%, 30%, 35%, 40%, 45%, 50%, 55%, 60%, 65%, 70%, 75%, 80%, 85%, 90%, 95%, 96%, 97%, 98%, 99%, 99.5% or more of at least one biological activity of the full-length polypeptide (e.g., the ability to up- or down-regulate gene expression). In some embodiments, the functional fragment actually has a higher level of at least one biological activity of the full-length polypeptide.

As used herein, the term “modified,” as applied to a polynucleotide or polypeptide sequence, refers to a sequence that differs from a wild-type sequence due to one or more deletions, additions, substitutions, or any combination thereof. Modified sequences may also be referred to as “modified variant(s).”

The terms “immunogen” and “antigen” are used interchangeably herein and mean any compound (including polypeptides) to which a cellular and/or humoral immune response can be directed. In particular embodiments, an immunogen or antigen can induce a protective immune response against the effects of dengue virus infection.

A “vector” refers to a compound used as a vehicle to carry foreign genetic material into another cell, where it can be replicated and/or expressed. A cloning vector containing foreign nucleic acid is termed a recombinant vector. Examples of nucleic acid vectors are plasmids, viral vectors, cosmids, expression cassettes, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs or expression cassettes) are for the expression of the exogenous gene in the target cell, and generally have a promoter sequence that drives expression of the exogenous gene. Insertion of a vector into the target cell is referred to transformation or transfection for bacterial and eukaryotic cells, although insertion of a viral vector is often called transduction. The term “vector” may also be used in general to describe items to that serve to carry foreign genetic material into another cell, such as, but not limited to, a transformed cell or a nanoparticle.

As used herein, by “isolate” or “purify” (or grammatical equivalents) a vector, it is meant that the vector is at least partially separated from at least some of the other components in the starting material.

“Effective amount” as used herein refers to an amount of a vector, nucleic acid, epitope, polypeptide, cell, particle, VLP, composition or formulation of the invention that is sufficient to produce a desired effect, which can be a therapeutic and/or beneficial effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation.

The term “immunogenic amount” or “effective immunizing dose,” as used herein, unless otherwise indicated, means an amount or dose sufficient to induce an immune response (which can optionally be a protective response) in the treated subject that is greater than the inherent immunity of non-immunized subjects. An immunogenic amount or effective immunizing dose in any particular context can be routinely determined using methods known in the art.

The terms “vaccine,” “vaccination” and “immunization” are well-understood in the art, and are used interchangeably herein. For example, the terms vaccine, vaccination or immunization can be understood to be a process or composition that increases a subject's immune reaction to an immunogen (e.g., by providing an active immune response), and therefore its ability to resist, overcome and/or recover from infection (i.e., a protective immune response).

By the terms “treat,” “treating” or “treatment of” (and grammatical variations thereof) it is meant that the severity of the subject's condition is reduced, at least partially improved or ameliorated and/or that some alleviation, mitigation or decrease in at least one clinical symptom is achieved and/or there is a delay in the progression of the disease or disorder. In representative embodiments, the terms “treat,” “treating” or “treatment of” (and grammatical variations thereof) refer to a reduction in the severity of viremia and/or a delay in the progression of viremia, with or without other signs of clinical disease.

A “treatment effective” amount as used herein is an amount that is sufficient to treat (as defined herein) the subject. Those skilled in the art will appreciate that the therapeutic effects need not be complete or curative, as long as some benefit is provided to the subject.

The term “prevent,” “preventing” or “prevention of” (and grammatical variations thereof) refer to prevention and/or delay of the onset and/or progression of a disease, disorder and/or a clinical symptom(s) in a subject and/or a reduction in the severity of the onset and/or progression of the disease, disorder and/or clinical symptom(s) relative to what would occur in the absence of the methods of the invention. In representative embodiments, the terms “prevent,” “preventing” or “prevention of” (and grammatical variations thereof) refer to prevention and/or delay of the onset and/or progression of viremia in the subject, with or without other signs of clinical disease. The prevention can be complete, e.g., the total absence of the disease, disorder and/or clinical symptom(s). The prevention can also be partial, such that the occurrence of the disease, disorder and/or clinical symptom(s) in the subject and/or the severity of onset and/or the progression is less than what would occur in the absence of the present invention.

A “prevention effective” amount as used herein is an amount that is sufficient to prevent (as defined herein) the disease, disorder and/or clinical symptom in the subject. Those skilled in the art will appreciate that the level of prevention need not be complete, as long as some benefit is provided to the subject.

The efficacy of treating and/or preventing dengue virus infection by the methods of the present invention can be determined by detecting a clinical improvement as indicated by a change in the subject's symptoms and/or clinical parameters (e.g., viremia), as would be well known to one of skill in the art.

Unless indicated otherwise, the terms “protect,” “protecting,” “protection” and “protective” (and grammatical variations thereof) encompass both methods of preventing and treating dengue virus infection in a subject, whether against one or multiple strains, genotypes or serotypes of dengue virus.

The terms “protective” immune response or “protective” immunity as used herein indicates that the immune response confers some benefit to the subject in that it prevents or reduces the incidence and/or severity and/or duration of disease or any other manifestation of infection. For example, in representative embodiments, a protective immune response or protective immunity results in reduced viremia, whether or not accompanied by clinical disease. Alternatively, a protective immune response or protective immunity may be useful in the therapeutic treatment of existing disease.

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMNUNOLOGY: BASIC PROCESSES 117 (Joseph A. Bellanti ed., 1985). Alternatively stated, an active immune response is mounted by the host after exposure to immunogens by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold, and/or at least about 5%, 10%, 15%, 20%, 25%, 30%, 35%, 40%, 45%, 50%, 55%, 60%, 65%, 70%, 75%, 80%, 85%, 90%, 95%, or 100% or more, or any value or range therein.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts may be referred to as “transcription products” and encoded polypeptides may be referred to as “translation products.” Transcripts and encoded polypeptides may be collectively referred to as “gene products.” If the polynucleotide is derived from genomic DNA, expression may include splicing of the mRNA in a eukaryotic cell. The expression product itself, e.g., the resulting nucleic acid or protein, may also be said to be “expressed.” An expression product can be characterized as intracellular, extracellular, or secreted. The term “intracellular” means something that is inside a cell. The term “extracellular” means something that is outside a cell. A substance is “secreted” by a cell if it appears in significant measure outside the cell, from somewhere on or inside the cell.

A “subject” of the invention includes any animal susceptible to dengue virus infection. Such a subject is generally a mammalian subject (e.g., a laboratory animal such as a rat, mouse, guinea pig, rabbit, primates, etc.), a farm or commercial animal (e.g., a cow, horse, goat, donkey, sheep, etc.), or a domestic animal (e.g., cat, dog, ferret, etc.). In particular embodiments, the subject is a primate subject, a non-human primate subject (e.g., a chimpanzee, baboon, monkey, gorilla, etc.) or a human. Subjects of the invention can be a subject known or believed to be at risk of infection by a flavivirus (e.g., dengue virus). Alternatively, a subject according to the invention can also include a subject not previously known or suspected to be infected by a flavivirus or in need of treatment for flavivirus infection.

Subjects may be treated for any purpose, such as for eliciting a protective immune response or for eliciting the production of antibodies in that subject, which antibodies can be collected and used for other purposes such as research or diagnostic purposes or for administering to other subjects to produce passive immunity therein, etc.

Subjects include males and/or females of any age, including neonates, juvenile, mature and geriatric subjects. With respect to human subjects, in representative embodiments, the subject can be an infant (e.g., less than about 12 months, 10 months, 9 months, 8 months, 7 months, 6 months, or younger), a toddler (e.g., at least about 12, 18 or 24 months and/or less than about 36, 30 or 24 months), or a child (e.g., at least about 1, 2, 3, 4 or 5 years of age and/or less than about 14, 12, 10, 8, 7, 6, 5, or 4 years of age). In embodiments of the invention, the subject is a human subject that is from about 0 to 3, 4, 5, 6, 9, 12, 15, 18, 24, 30, 36, 48 or 60 months of age, from about 3 to 6, 9, 12, 15, 18, 24, 30, 36, 48 or 60 months of age, from about 6 to 9, 12, 15, 18, 24, 30, 36, 48 or 60 months of age, from about 9 to 12, 15, 18, 24, 30, 36, 48 or 60 months of age, from about 12 to 18, 24, 36, 48 or 60 months of age, from about 18 to 24, 30, 36, 48 or 60 months of age, or from about 24 to 30, 36, 48 or 60 months of age.

A “subject in need” of the methods of the invention can be a subject known to be, or suspected of being, infected with, or at risk of being infected with, a flavivirus (e.g., dengue virus).

The term “administering” or “administration” of a composition of the present invention to a subject includes any route of introducing or delivering to a subject a compound to perform its intended function (e.g., for use as a vaccine antigen). Administration includes self-administration and the administration by another.

A “sample” or “biological sample” of this invention can be any biological material, such as a biological fluid, an extract from a cell, an extracellular matrix isolated from a cell, a cell (in solution or bound to a solid support), a tissue, a tissue homogenate, and the like as are well known in the art.

### Compositions

A unique hallmark of Dengue virus (DENV), antibody dependent enhancement (ADE) causes deadly DENV secondary infection, complicates the identification of correlates of protection, and negatively impacts the safety and efficacy of DENV vaccines. ADE has been linked to antibodies targeting the pre-Membrane (prM) protein and the fusion loop (FL) of Envelope (E) protein, a motif which is completely conserved in mosquito-borne flaviviruses and required for DENV infection.

ADE remains a major challenge for DENV vaccine development (Rey et al. 2018 EMBO Rep 19:206-224). Currently, there is one approved DENV vaccine, Dengvaxia. However, it is only recommended for children aged 9-16 with previous DENV infection and is contraindicated for naïve individuals; in naïve children, vaccination was associated with negative outcomes (Wilder-Smith et al. 2019 Lancet Infect Dis 19, e31-e38; Dengue vaccine: WHO position paper, September 2018—Recommendations. Vaccine 37, 4848-4849 (2019)). Other DENV vaccines have been tested or are currently undergoing clinical trial, but thus far no others have been approved for use (Izmirly et al. 2020 Front Immunol 11:1055). The majority of DENV vaccine platforms use live virus strains, with a mixture of all four serotypes. However, creating formulations that will elicit a balanced response has been challenging (Rabaa et al. 2017 Elife 6:1-22). Additionally, there is evidence that lab-grown strains are very different from patient derived DENVs in maturity and antigenicity (Raut et al. 2019 PNAS 116:227-232).

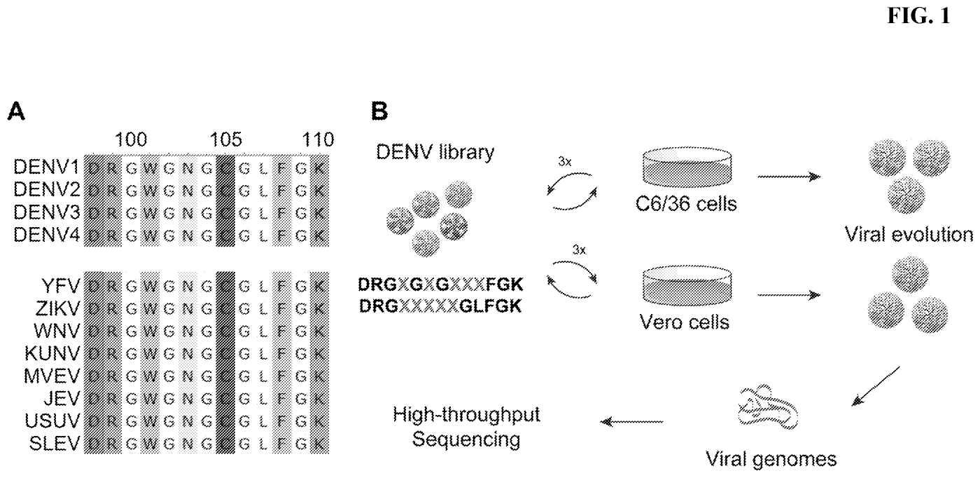

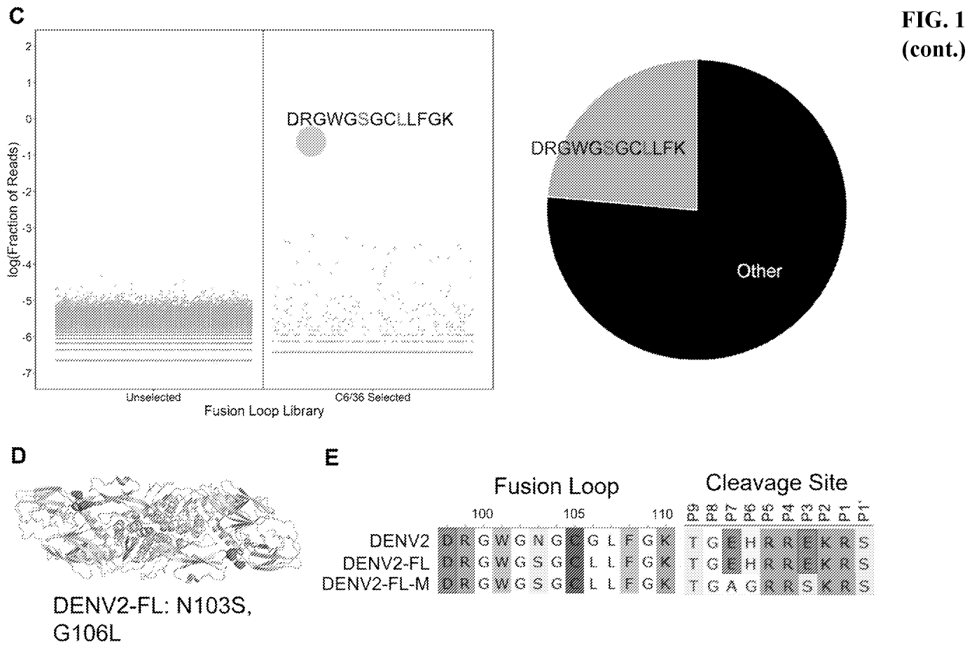

Antibodies targeting the fusion loop have been reported to neutralize lab and patient strains with differing strength, as well as to facilitate Fc-receptor uptake and therefore ADE (Lai et al. 2013 PLoS Negl Trop Dis 7:1-11; Beltramello et al. 2010 cell Host Microbe 8:271-283; Costin et al. 2013 J Virol 87:52-66). The fusion loop is contained in the DENV Envelope (E) domain II (EDII) and is involved in monomer-monomer contacts with EDIII (Modis et al. 2004 Nature 427:313-319). During the DENV infection cycle, low pH triggers a conformational change in the E protein (Klein et al. 2013 J Virol 87:2287-2293). The structure of the virion rearranges, and individual monomers form a trimer with all three fusion loops in the same orientation, ready for membrane fusion. The motif is highly conserved, with 100% amino acid conservation in all DENV serotypes and other mosquito-borne flaviviruses, including Yellow fever virus (YFV), Zika virus (ZIKV), West Nile virus (WNV), Kunjin virus (KUNV), Murray Valley encephalitis virus (MVEV), Japanese encephalitis virus (JEV), Usutu virus (USUV), and Saint Louis encephalitis virus (SLEV) (FIG. 1 panel A).

The present invention is based on the novel engineering of a viable variant with a mutated but functional fusion loop which is insensitive to ADE-prone FL-targeting monoclonal antibodies but retains sensitivity to other protective type-specific (TS) and cross-reactive (CR) antibodies (Abs).

One aspect of the present invention provides a recombinant flavivirus E glycoprotein comprising a flavivirus E glycoprotein backbone comprising a fusion loop epitope, and one or more of the following amino acid substitutions in the fusion loop epitope, wherein the numbering corresponds to the amino acid sequence of SEQ ID NO:1[wt DV2 E]: 103S and/or 106L. In some embodiments, the recombinant flavivirus E glycoprotein comprising a flavivirus E glycoprotein backbone may comprise a fusion loop epitope comprising the amino acid substitutions 103S and 106L.

As used herein, the term “amino acid” or “amino acid residue” encompasses any naturally occurring amino acid, modified forms thereof, and synthetic amino acids. Naturally occurring, levorotatory (L-) amino acids as shown in Table 1.

Conservative amino acid substitutions are known in the art. In particular embodiments, a conservative amino acid substitution includes substitutions within one or more of the following groups: glycine, alanine; valine, isoleucine, leucine; aspartic acid, glutamic acid; asparagine, glutamine; serine, threonine; lysine, arginine; and/or phenylalanine, tyrosine.

Alternatively, the amino acid can be a modified amino acid residue (nonlimiting examples are shown in Table 2) and/or can be an amino acid that is modified by post-translation modification (e.g., acetylation, amidation, formylation, hydroxylation, methylation, phosphorylation or sulfatation). In addition, the non-naturally occurring amino acid can be an “unnatural” amino acid such as described by Wang et al., Annu Rev Biophys Biomol Struct. 35:225-49 (2006)). These unnatural amino acids can advantageously be used to chemically link molecules of interest to a virus particle.

In some embodiments, an amino acid residue is substituted adjacent to an insertion. In some embodiments, a substitution may comprise a deletion of one or more amino acid residues. The substitution(s) and residue positions may be described in one or more ways that are redundant in generating the same resultant amino acid sequence. Thus, a disclosure of one such description is herein considered a disclosure of each and inclusive of all such redundant disclosures. Thus, wherein the resultant amino acid sequence is identical, any disclosure provided herein describing one way to produce the resultant amino acid sequence is considered a disclosure of each and inclusive of all such redundant disclosures.

In some embodiments, the recombinant flavivirus E glycoprotein of the invention comprising a flavivirus E glycoprotein backbone may comprise a fusion loop epitope further comprising one or more amino acid substitution(s) in position 101, 102, 104, 105, and/or 107.

In some embodiments, the amino acid substitutions may comprise a substitution of the wildtype amino acid residue with a lysine (K), histidine (H), arginine (R), tyrosine (Y), serine (S), glutamic acid (E), valine (V), glycine (G), aspartic acid (D), methionine (M), glutamine (Q), asparagine (N), alanine (A), isoleucine (I), proline (P), phenylalanine (F), threonine (T), leucine (L), tryptophan (W), cysteine (C), or any derivative thereof at amino acid position 101, 102, 104, 105, and/or 107 of the backbone E glycoprotein.

In some embodiments, the one or more amino acid substitution(s) may comprise 101W, 102G, 104G, 105C, and/or 107L, in any combination.

In some embodiments, the recombinant E glycoprotein may have reduced ability to stimulate targeting and/or neutralization by antibody-dependent enhancement (ADE)-associated anti-flavivirus antibodies, e.g., at least about 10%, 20%, 30%, 40%, 50%, 60%, 70%, 80%, 90%, 91%, 92%, 93%, 94%, 95%, 96%, 97%, 98%, 99%, or 100% reduced ability to targeting and/or neutralization by ADE-associated anti-flavivirus antibodies as compared to an unmodified E glycoprotein of a corresponding wildtype flavivirus, or any value or range therein. For example, in some embodiments, a recombinant E glycoprotein of the present invention may have reduced ability to stimulate targeting and/or neutralization by ADE-associated anti-flavivirus antibodies at least about 10% to about 100%, about 20% to about 80%, about 40% to about 50%, or at least about 10%, about 20%, about 50%, about 80% or about 95% as compared to an unmodified E glycoprotein of a corresponding wildtype flavivirus.

In some embodiments, the recombinant E glycoprotein may be less sensitive to targeting and/or neutralization by fusion loop targeting anti-flavivirus antibodies, e.g., at least about 10%, 20%, 30%, 40%, 50%, 60%, 70%, 80%, 90%, 91%, 92%, 93%, 94%, 95%, 96%, 97%, 98%, 99%, or 100% less sensitive to targeting and/or neutralization by fusion loop targeting anti-flavivirus antibodies as compared to an unmodified E glycoprotein of a corresponding wildtype flavivirus, or any value or range therein. For example, in some embodiments, a recombinant E glycoprotein of the present invention may be less sensitive to targeting and/or neutralization by fusion loop targeting anti-flavivirus antibodies at least about 10% to about 100%, about 20% to about 80%, about 40% to about 50%, or at least about 10%, about 20%, about 50%, about 80% or about 95% as compared to an unmodified E glycoprotein of a corresponding wildtype flavivirus.

The flavivirus E glycoprotein backbone of the present invention may be from any flavivirus known or later discovered. For example, in some embodiments, the E glycoprotein backbone may comprise a backbone of a flavivirus such as, but not limited to, dengue virus (DENV, also referred to as DV), zika virus (ZIKV), West Nile virus (WNV), Japanese encephalitis virus (JEV), yellow fever virus (YFV), tick-borne encephalitis virus (TBEV), Kyasanur Forest disease virus (KFDV), Kunjin virus (KUN), Murray Valley encephalitis virus (MVEV), St. Louis encephalitis virus (SLEV), Powassan virus (POW), Usutu virus (USUV), and/or any derivative thereof. In some embodiments, the E glycoprotein backbone may be a backbone of dengue virus serotype 1, 2, 3, 4, or any derivative thereof, e.g., a modified DV-1, -2, -3, -4, a chimeric DV, a fully synthetic DV, and the like.

A DENV1 backbone may comprise the amino acid sequence of any DENV1 genotype and/or strain and/or isolate currently known or as yet identified and/or isolated. Non-limiting examples of DENV1 genotypes, strains, and/or isolates include Genotypes I, II, III, IV, and V, strains Western Pacific 1974, and GenBank® Accession No. U88535.1.

A DENV3 backbone may comprise the amino acid sequence of any DENV3 genotype and/or strain and/or isolate currently known or as yet identified and/or isolated. Non-limiting examples of DENV3 genotypes, strains, and/or isolates include Genotype I, II, III, IV, strains such as Sri Lanka 1989, Indonesia 1982, Thailand 1995, Cuba 2002, and Puerto Rico 1977, and GenBank® Accession Nos. JQ411814.1 (“UNC3001”), DQ401690.1, AY676376, AY02031, and AY146761.

A DENV2 backbone may comprise the amino acid sequence of any DENV2 genotype and/or strain and/or isolate currently known or as yet identified and/or isolated. Non-limiting examples of DENV2 genotypes, strains, and/or isolates include Genotypes Cosmopolitan, Asian-American, Asian I, and Asian II, strains such as S-16803 (GenBank® Accession No. GU289914.1), India 2001 (GenBank® Accession No. DQ448237b), Puerto Rico 2003 (GenBank® Accession No. EU687235b), USA 2009 (GenBank® Accession No. HQ541798b), Indonesia 2016 (GenBank® Accession No. MH173162), Thailand 2016 (GenBank® Accession No. LC410185b), and GenBank® Accession No. NC_001474.2.

A DENV4 backbone may comprise the amino acid sequence of any DENV4 genotype and/or strain and/or isolate currently known or as yet identified and/or isolated. Non-limiting examples of DENV4 genotypes, strains, and/or isolates include Genotypes I, IIa, IIb, III, IV, and V and strains such as GenBank® Accession Nos. KF543272, JN832541, FJ882599, KJ160504.1, AY618940, AF231724, and JF262783.

In some embodiments, the fusion loop epitope of the present invention comprises the following amino acid sequence in positions 98-110, wherein the numbering corresponds to the amino acid sequence of SEQ ID NO:1: DRGWGSGCLLFGK (SEQ ID NO:10).

In some embodiments, the recombinant flavivirus E glycoprotein backbone of the present invention may comprise an amino acid sequence at least 70% identical (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical) to the amino acid sequence of SEQ ID NO:3.

[wt DENV1 E, GenBank ® U88535.1]
SEQ ID NO: 3
MRCVGIGNRDFVEGLSGATWVDVVLEHGSCVTTMAKDKPTLDIELLKTEVTNPAVL
RKLCIEAKISNTTTDSRCPTQGEATLVEEQDTNFVCRRTFVDRGWGNGCGLFGKGSL
ITCAKFKCVTKLEGKIVQYENLKYSVIVTVHTGDQHQVGNETTEHGTTATITPQAPTS
EIQLTDYGALTLDCSPRTGLDFNEMVLLTMEKKSWLVHKQWFLDLPLPWTSGASTS
QETWNRQDLLVTFKTAHAKKQEVVVLGSQEGAMHTALTGATEIQTSGTTTIFAGHL
KCRLKMDKLTLKGMSYVMCTGSFKLEKEVAETQHGTVLVQVKYEGTDAPCKIPFSS
QDEKGVTQNGRLITANPIVTDKEKPVNIEAEPPFGESYIVVGAGEKALKLSWFKKGSS
IGKMFEATARGARRMAILGDTAWDFGSIGGVFTSVGKLIHQIFGTAYGVLFSGVSWT
MKIGIGILLTWLGLNSRSTSLSMTCIAVGMVTLYLGVMVQADSGCVINWKGRELKC
GSGIFVTNEVHTWTEQYKFQADSPKRLSAAIGKAWEEGVCGIRSATRLENIMWKQIS
NELNHILLENDMKFTVVVGDVSGILAQGKKMIRPQPMEHKYSWKSWGKAKIIGADV
QNTTFIIDGPNTPECPDNQRAWNIWEVE

In some embodiments, the recombinant flavivirus E glycoprotein of the present invention may comprise, consist essentially of, or consist of an amino acid sequence at least 70% identical (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical) to the amino acid sequence of SEQ ID NO:7.

[substituted DENV1 E]
SEQ ID NO: 7
MRCVGIGNRDFVEGLSGATWVDVVLEHGSCVTTMAKDKPTLDIELLKTEVTNPAVL
RKLCIEAKISNTTTDSRCPTQGEATLVEEQDTNFVCRRTFVDRGWGSGCLLFGKGSLI
TCAKFKCVTKLEGKIVQYENLKYSVIVTVHTGDQHQVGNETTEHGTTATITPQAPTS
EIQLTDYGALTLDCSPRTGLDFNEMVLLTMEKKSWLVHKQWFLDLPLPWTSGASTS
QETWNRQDLLVTFKTAHAKKQEVVVLGSQEGAMHTALTGATEIQTSGTTTIFAGHL
KCRLKMDKLTLKGMSYVMCTGSFKLEKEVAETQHGTVLVQVKYEGTDAPCKIPFSS
QDEKGVTQNGRLITANPIVTDKEKPVNIEAEPPFGESYIVVGAGEKALKLSWFKKGSS
IGKMFEATARGARRMAILGDTAWDFGSIGGVFTSVGKLIHQIFGTAYGVLFSGVSWT
MKIGIGILLTWLGLNSRSTSLSMTCIAVGMVTLYLGVMVQADSGCVINWKGRELKC
GSGIFVTNEVHTWTEQYKFQADSPKRLSAAIGKAWEEGVCGIRSATRLENIMWKQIS
NELNHILLENDMKFTVVVGDVSGILAQGKKMIRPQPMEHKYSWKSWGKAKIIGADV
QNTTFIIDGPNTPECPDNQRAWNIWEVE

In some embodiments, the recombinant flavivirus E glycoprotein backbone of the present invention may comprise an amino acid sequence at least 70% identical (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical) to the amino acid sequence of SEQ ID NO:1.

[wt DENV2 E, GenBank ® GU289914]
SEQ ID NO: 1
MRCIGISNRDFVEGVSGGSWVDIVLEHGSCVTTMAKNKPTLDFELIKTEA
KQPATLRKYCIEAKLTNTTTESRCPTQGEPSLNEEQDKRFVCKHSMVDRG
WGNGCGLFGKGGIVTCAMFTCKKNMEGKVVQPENLEYTIVVTPHSGEEHA
VGNDTGKHGKEIKVTPQSSITEAELTGYGTVTMECSPRTGLDFNEMVLLQ
MENKAWLVHRQWFLDLPLPWLPGADTQGSNWIQKETLVTFKNPHAKKQDV
VVLGSQEGAMHTALTGATEIQMSSGNLLFTGHLKCRLRMDKLQLKGMSYS
MCTGKFKVVKEIAETQHGTIVIRVQYEGDGSPCKIPFEIMDLEKRHVLGR
LITVNPIVTEKDSPVNIEAEPPFGDSYIIIGVDPGQLKLNWFKKGSSIGQ
MFETTMRGAKRMAILGDTAWDFGSLGGVFTSIGKALHQVFGAIYGAAFSG
VSWTMKILIGVIITWIGMNSRSTSLSVSLVLVGIVTLYLGVMVQA.

In some embodiments, the recombinant flavivirus E glycoprotein of the present invention may comprise, consist essentially of, or consist of an amino acid sequence at least 70% identical (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical) to the amino acid sequence of SEQ ID NO:6.

[substituted DENV2 E]
SEQ ID NO: 6
MRCIGISNRDFVEGVSGGSWVDIVLEHGSCVTTMAKNKPTLDFELIKTEA
KQPATLRKYCIEAKLTNTTTESRCPTQGEPSLNEEQDKRFVCKHSMVDRG
WGSGCLLFGKGGIVTCAMFTCKKNMEGKVVQPENLEYTIVVTPHSGEEHA
VGNDTGKHGKEIKVTPQSSITEAELTGYGTVTMECSPRTGLDFNEMVLLQ
MENKAWLVHRQWFLDLPLPWLPGADTQGSNWIQKETLVTFKNPHAKKQDV
VVLGSQEGAMHTALTGATEIQMSSGNLLFTGHLKCRLRMDKLQLKGMSYS
MCTGKFKVVKEIAETQHGTIVIRVQYEGDGSPCKIPFEIMDLEKRHVLGR
LITVNPIVTEKDSPVNIEAEPPFGDSYIIIGVDPGQLKLNWFKKGSSIGQ
MFETTMRGAKRMAILGDTAWDFGSLGGVFTSIGKALHQVFGAIYGAAFSG
VSWTMKILIGVIITWIGMNSRSTSLSVSLVLVGIVTLYLGVMVQA.

In some embodiments, the recombinant flavivirus E glycoprotein backbone of the present invention may comprise an amino acid sequence at least 70% identical (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical) to the amino acid sequence of SEQ ID NO:4.

[wt DENV3 E, GenBank ® JQ411814.1]
SEQ ID NO: 4
MRCVGIGNRDFVEGLSGATWVDVVLEHGGCVTTMAKNKPTLDIELQKTEA
TQLATLRKLCIEGKITNITTDSRCPTQGEAVLPEEQDQNYVCKHTYVDRG
WGNGCGLFGKGSLVTCAKFQCLEPIEGKVVQYENLKYTVIITVHTGDQHQ
VGNETQGVTAEITPQASTTEAILPEYGTLGLECSPRTGLDFNEMILLTMK
NKAWMVHRQWFFDLPLPWTSGATTETPTWNRKELLVTFKNAHAKKQEVVV
LGSQEGAMHTALTGATEIQNSGGTSIFAGHLKCRLKMDKLELKGMSYAMC
TNTFVLKKEVSETQHGTILIKVEYKGEDAPCKIPFSTEDGQGKAHNGRLI
TANPVVTKKEEPVNIEAEPPFGESNIVIGIGDNALKINWYKKGSSIGKMF
EATARGARRMAILGDTAWDFGSVGGVLNSLGKMVHQIFGSAYTALFSGVS
WVMKIGIGVLLTWIGLNSKNTSMSFSCIAIGIITLYLGAVVQADMGCVIN
WKGKELKCGSGIFVTNEVHTWTEQYKFQADSPKRLATAIAGAWENGVCGI
RSTTRMENLLWKQIANELNYILWENNIKLTVVVGDTIGVLEQGKRTLTPQ
PMELKYSWKTWGKAKIVTAETQNSSFIIDGPNTPECPSASRAWNVWEVE

In some embodiments, the recombinant flavivirus E glycoprotein of the present invention may comprise, consist essentially of, or consist of an amino acid sequence at least 70% identical (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical) to the amino acid sequence of SEQ ID NO:8.

[substituted DENV3 E]
SEQ ID NO: 8
MRCVGIGNRDFVEGLSGATWVDVVLEHGGCVTTMAKNKPTLDIELQKTEA
TQLATLRKLCIEGKITNITTDSRCPTQGEAVLPEEQDQNYVCKHTYVDRG
WGSGCLLFGKGSLVTCAKFQCLEPIEGKVVQYENLKYTVIITVHTGDQHQ
VGNETQGVTAEITPQASTTEAILPEYGTLGLECSPRTGLDFNEMILLTMK
NKAWMVHRQWFFDLPLPWTSGATTETPTWNRKELLVTFKNAHAKKQEVVV
LGSQEGAMHTALTGATEIQNSGGTSIFAGHLKCRLKMDKLELKGMSYAMC
TNTFVLKKEVSETQHGTILIKVEYKGEDAPCKIPFSTEDGQGKAHNGRLI
TANPVVTKKEEPVNIEAEPPFGESNIVIGIGDNALKINWYKKGSSIGKMF
EATARGARRMAILGDTAWDFGSVGGVLNSLGKMVHQIFGSAYTALFSGVS
WVMKIGIGVLLTWIGLNSKNTSMSFSCIAIGIITLYLGAVVQADMGCVIN
WKGKELKCGSGIFVTNEVHTWTEQYKFQADSPKRLATAIAGAWENGVCGI
RSTTRMENLLWKQIANELNYILWENNIKLTVVVGDTIGVLEQGKRTLTPQ
PMELKYSWKTWGKAKIVTAETQNSSFIIDGPNTPECPSASRAWNVWEVE

In some embodiments, the recombinant flavivirus E glycoprotein backbone of the present invention may comprise an amino acid sequence at least 70% identical (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical) to the amino acid sequence of SEQ ID NO:5.

[wt DENV4 E, GenBank ® KJ160504.1]
SEQ ID NO: 5
MRCVGVGNRDFVEGVSGGAWVDLVLEHGGCVTTMAQGKPTLDFELTKTTA
KEVALLRTYCIEASISNITTATRCPTQGEPYLKEEQDQQYICRRDVVDRG
WGNGCGLFGKGGVVTCAKFSCSGKITGNLVQIENLEYTVVVTVHNGDTHA
VGNDTSNHGVTATITPRSPSVEVKLPDYGELTLDCEPRSGIDFNEMILMK
MKKKTWLVHKQWFLDLPLPWTAGADTSEVHWNYKERMVTFKVPHAKRQDV
TVLGSQEGAMHSALAGATEVDSGDGNHMFAGHLKCKVRMEKLRIKGMSYT
MCSGKFSIDKEMAETQHGTTVVKVKYEGAGAPCKVPIEIRDVNKEKVVGR
VISSTPLAENTNSVTNIELEPPFGDSYIVIGVGNSALTLHWFRKGSSIGK
MFESTYRGAKRMAILGETAWDFGSVGGLFTSLGKAVHQVFGSVYTTMFGG
VSWMIRILIGFLVLWIGTNSRNTSMAMTCIAVGGITLFLGFTVQA

In some embodiments, the recombinant flavivirus E glycoprotein of the present invention may comprise, consist essentially of, or consist of an amino acid sequence at least 70% identical (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical) to the amino acid sequence of SEQ ID NO:9.

[substituted DENV4 E]
SEQ ID NO: 9
MRCVGVGNRDFVEGVSGGAWVDLVLEHGGCVTTMAQGKPTLDFELTKTTA
KEVALLRTYCIEASISNITTATRCPTQGEPYLKEEQDQQYICRRDVVDRG
WGSGCLLFGKGGVVTCAKFSCSGKITGNLVQIENLEYTVVVTVHNGDTHA
VGNDTSNHGVTATITPRSPSVEVKLPDYGELTLDCEPRSGIDFNEMILMK
MKKKTWLVHKQWFLDLPLPWTAGADTSEVHWNYKERMVTFKVPHAKRQDV
TVLGSQEGAMHSALAGATEVDSGDGNHMFAGHLKCKVRMEKLRIKGMSYT
MCSGKFSIDKEMAETQHGTTVVKVKYEGAGAPCKVPIEIRDVNKEKVVGR
VISSTPLAENTNSVTNIELEPPFGDSYIVIGVGNSALTLHWFRKGSSIGK
MFESTYRGAKRMAILGETAWDFGSVGGLFTSLGKAVHQVFGSVYTTMFGG
VSWMIRILIGFLVLWIGTNSRNTSMAMTCIAVGGITLFLGFTVQA

The amino acid residue positions of the substitutions that can be made to produce the desired recombinant E glycoproteins of the present invention can be readily determined by one of ordinary skill in the art according to the teachings herein and according to protocols well known in the art. The amino acid residue numbering provided in the amino acid sequences set forth here is based on the reference sequence of DENV2 wild type E glycoprotein, as provided herein (SEQ ID NO:1). However it would be readily understood by one of ordinary skill in the art that the equivalent amino acid positions in other flavivirus E glycoproteins can be readily identified and employed in the production of the recombinant E glycoproteins of this invention.

It would be understood that the modifications described above provide multiple examples of how the amino acid sequences described herein can be obtained and that, due to the degeneracy of the amino acid codons, numerous other modifications can be made to a nucleotide sequence encoding a E glycoprotein or fragment thereof to obtain the desired amino acid sequence. The present invention provides additional non limiting examples of nucleic acids and/or polypeptides of this invention that can be used in the compositions and methods described herein in the SEQUENCES section provided herein.

The present invention further provides an isolated nucleic acid molecule encoding the recombinant flavivirus E glycoprotein of this invention. In some embodiments, a nucleic acid molecule of this invention may be a cDNA molecule. In some embodiments, a nucleic acid molecule of this invention may be an mRNA molecule, e.g., an mRNA-based prophylactic and/or therapeutic immunogen (e.g., vaccine), e.g., an mRNA-lipid nanoparticle (mRNA-LNP) molecule.

In some embodiments, an isolated nucleic acid molecule of the present invention may further encode a modified flavivirus prM glycoprotein.

Proteolytic cleavage of viral membrane fusion proteins is a strategy for temporal or spatial control of virus infection, ultimately affecting tropism and transmission (White et al. 2008 Crit. Rev. Biochem. Mol. Biol. 43:189-219). In DENV, maturation is controlled by furin cleavage of the prM glycoprotein. Furin is a ubiquitously expressed host serine protease in the trans-Golgi network (TGN). Cleavage of prM releases the pr portion from the virion, and triggers a rotation and collapse of E glycoprotein to form the mature virion. In addition to conformational change in proteins, lipids may also play a role in stabilizing the mature DENV structure.

In some embodiments, a modified flavivirus prM glycoprotein of the present invention may comprise a furin cleavage site with enhanced cleavability by a furin enzyme (e.g., as compared to an unsubstituted furin cleavage site in a corresponding wildtype flavivirus prM glycoprotein).

In some embodiments, a modified flavivirus prM glycoprotein of the present invention may comprise a furin cleavage site comprising one or more substitution at amino acid position 85, 86, 87, and/or 89, wherein the numbering corresponds to the amino acid sequence of SEQ ID NO:12 [wt DENV2 prM].

[DV2-prM] GenBank: GU289914.1
SEQ ID NO: 12
FHLTTRNGEPHMIVSRQEKGKSLLFKTEDGVNMCTLMAMDLGELCEDTIT
YNCPLLRQNEPEDIDCWCNSTSTWVTYGTCTTTGEHRREKRSVALVPHVG
MGLETRTETWMSSEGAWKHAQRIETWILRHPGFTIMAAILAYTIGTTHFQ
RALIFILLTAVAPSMT.

In some embodiments, the substitution of an amino acid residue that is more basic comprises 89K (e.g., E89K, D89K). In some embodiments, the substitution of an amino acid residue that is more basic comprises 89N (e.g., E89N, D89N). In some embodiments, the substitution of an amino acid residue that is more basic comprises 89Y (e.g., E89Y, D89Y). In some embodiments, the substitution of an amino acid residue that is more basic comprises 89S (e.g., E89S, D89S).

In some embodiments, the one or more amino acid substitutions at positions 87, 86, and 85 comprise 85R, 85A, 85S, 86A, 86G, 86K, 87Q, 87R, 87M, or any combination thereof.

In some embodiments, the furin cleavage site has enhanced cleavability by a furin enzyme, e.g., as compared to an unsubstituted furin cleavage site in a corresponding wildtype flavivirus prM glycoprotein.

In some embodiments, the furin cleavage site cleavability is about 1.5-fold enhanced, e.g., about 1.5-, 2-, 2.5-, 3-, 3.5-, 4-, 4.5-, 5-, 5.5-, 6-, 6.5-, 7-, 7.5-, 8-, 8.5-, 9-, 9.5-, or 10-fold enhanced or higher, or any value or range therein. Enhancement of furin cleavage site cleavability may be measured by any standard method in the art, as will be understood by the skilled artisan upon review of the disclosures of the present invention. Non-limiting examples include use of Pi-Tou score and/or a ProP score, as described in Tian et al. 2012 Sci. Rep. 2:261, incorporated herein by reference in its entirety. For example, in some embodiments, the furin cleavage site cleavability is about 1.5-fold enhanced, e.g., about 1.5-, 2-, 2.5-, 3, 3.5-, 4-, 4.5-, 5-, 5.5-, 6-, 6.5-, 7-, 7.5-, 8-, 8.5-, 9-, 9.5-, or 10-fold enhanced or higher, or any value or range therein, as measured by relative Pi-Tou score of the substituted furin cleavage site as compared to the Pi-Tou score of an unsubstituted furin cleavage site in a corresponding wildtype flavivirus prM glycoprotein. In some embodiments, the furin cleavage site cleavability is enhanced about 1.5-fold to about 10-fold, about 2-fold to about 5-fold, about 1.5-fold to about 20-fold, or about 1.5-fold, about 2-fold, about 5-fold, about 10-fold, or about 20-fold or higher.

In some embodiments, the furin cleavage site has a Pi-Tou score of about 5 or higher, e.g., about 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, or higher, or any value or range therein. For example, in some embodiments, the furin cleavage site has a Pi-Tou score of about 5 to about 10, about 5 to about 20, about 6.5 to about 9, about 6.5 to about 15.5, about 10 to about 15.5, or about 5, about 6.5, about 9, about 10, about 15.5, or about 20.

The flavivirus prM glycoprotein of the present invention may be from any flavivirus known or later discovered, including but not limited to those previously disclosed herein for the E glycoprotein backbone. For example, in some embodiments, the prM glycoprotein may comprise a backbone of a flavivirus such as, but not limited to, dengue virus (DENV, also referred to as DV), zika virus (ZIKV), West Nile virus (WNV), Japanese encephalitis virus (JEV), yellow fever virus (YFV), tick-borne encephalitis virus (TBEV), Kyasanur Forest disease virus (KFDV), Kunjin virus (KUN), Murray Valley encephalitis virus (MVEV), St. Louis encephalitis virus (SLEV), Powassan virus (POW), Usutu virus (USUV), and/or any derivative thereof. In some embodiments, the prM glycoprotein may be a backbone of dengue virus serotype 1, 2, 3, 4, or any derivative thereof, e.g., a modified DV-1, -2, -3, -4, a chimeric DV, a fully synthetic DV, and the like.

In some embodiments, the flavivirus prM glycoprotein of the present invention may be any prM glycoprotein as described in PCT/US2022/020791, the disclosures of which are incorporated herein by reference.

In some embodiments, the furin cleavage site of the present invention may comprise, consist essentially of, or consist of the amino acid sequence of SEQ ID NO:11 in positions 83-92 of the wildtype (unmodified) prM glycoprotein, wherein the numbering corresponds to the amino acid sequence of SEQ ID NO:12 [wt DENV2 prM].

[modified prM cleavage site]
SEQ ID NO: 11
TGAGRRSKRS.

Also provided is a vector, plasmid or other nucleic acid construct comprising the isolated nucleic acid molecule of this invention.

A vector can be any suitable means for delivering a polynucleotide to a cell. A vector of this invention can be an expression vector that contains all of the genetic components required for expression of the nucleic acid in cells into which the vector has been introduced, as are well known in the art. The expression vector can be a commercial expression vector or it can be constructed in the laboratory according to standard molecular biology protocols. The expression vector can comprise viral nucleic acid including, but not limited to, poxvirus, vaccinia virus, adenovirus, retrovirus, alphavirus and/or adeno-associated virus nucleic acid. The nucleic acid molecule or vector of this invention can also be in a liposome or a delivery vehicle, which can be taken up by a cell via receptor-mediated or other type of endocytosis. The nucleic acid molecule of this invention can be in a cell, which can be a cell expressing the nucleic acid whereby a recombinant prM glycoprotein of this invention is produced in the cell (e.g., a host cell). In addition, the vector of this invention can be in a cell, which can be a cell expressing the nucleic acid of the vector whereby a recombinant E glycoprotein and/or prM glycoprotein of this invention is produced in the cell. It is also contemplated that the nucleic acid molecules and/or vectors of this invention can be present in a host organism (e.g., a transgenic organism), which expresses the nucleic acids of this invention and produces a recombinant prM glycoprotein of this invention. In some embodiments, the vector is a plasmid, a viral vector, a bacterial vector, an expression cassette, a transformed cell, or a nanoparticle. For example, in some embodiments a recombinant E glycoprotein and/or prM glycoprotein of the present invention may be used in combination (e.g., in scaffold(s) and/or conjugated with) other molecules such as, but not limited to, nanoparticles, e.g., as delivery devices.

Types of nanoparticles of this invention for use as a vector and/or delivery device include, but are not limited to, polymer nanoparticles such as PLGA-based, PLA-based, polysaccharide-based (dextran, cyclodextrin, chitosan, heparin), dendrimer, hydrogel; lipid-based nanoparticles such as lipid nanoparticles, lipid hybrid nanoparticles, liposomes, micelles; inorganics-based nanoparticles such as superparamagnetic iron oxide nanoparticles, metal nanoparticles, platin nanoparticles, calcium phosphate nanoparticles, quantum dots; carbon-based nanoparticles such as fullerenes, carbon nanotubes; and protein-based complexes with nanoscales. Types of microparticles of this invention include but are not limited to particles with sizes at micrometer scale that are polymer microparticles including but not limited to, PLGA-based, PLA-based, polysaccharide-based (dextran, cyclodextrin, chitosan, heparin), dendrimer, hydrogel; lipid-based microparticles such as lipid microparticles, micelles; inorganics-based microparticles such as superparamagnetic iron oxide microparticles, platin microparticles and the like as are known in the art. These particles may be generated and/or have materials be absorbed, encapsulated, or chemically bound through known mechanisms in the art.

In some embodiments, a nanoparticle vector of the present invention may be an mRNA lipid nanoparticle (mRNA-LNP), a nucleic acid vaccine (NAV), or other nucleic acid lipid nanoparticle compositions, such as described in U.S. Pat. Nos. 9,868,692; 9,950,065; 10,041,091; 10,576,146; 10,702,600; WO2015/164674; US2019/0351048; US2020/297634; WO2020/097548; and Buschmann et al. 2021 Vaccines 9(65); Laczkó et al. 2020 Immunity 53:724-732; and Pardi et al. 2018 Nat. Rev. Drug Discov. 17:261-279, the disclosures of each of which are incorporated herein by reference in their entireties.

It is known in the art that many attempts to produce flavivirus vaccines such as dengue virus vaccines result in the production of non-neutralizing antibodies, which may increase the likelihood of pathology upon subsequence exposure to natural infection or vaccine. Another approach to provide an engineered epitope is to deliver all or a portion of the flavivirus protein incorporated into another flavivirus particle or VLP. Portions of the flavivirus protein (e.g., prM glycoprotein, e.g., E glycoprotein, e.g., capsid protein) can be grafted into the relevant protein of the heterologous flavivirus backbone, e.g., to reduce the generation of non-neutralizing dengue virus antibodies to non-neutralizing epitopes present in the relevant flavivirus protein and/or other virus structural proteins.

Thus, a modified (e.g., recombinant and/or chimeric) flavivirus or flavivirus VLP can present the quaternary flavivirus epitope in proper conformation while reducing the generation of non-neutralizing antibodies to other portions of the flavivirus proteins that are not presented in the modified flavivirus or flavivirus VLP.

In some embodiments of the invention the individual and conformational epitopes of the flavivirus glycoproteins can be presented on a synthetic backbone or support structure so that the epitopes within the synthetic backbone or support structure mimic the conformation and arrangement of the epitopes within the structure of the source glycoprotein, virus particle or VLP.

In still further embodiments of the invention, the present invention provides peptide mimitopes (see, Meloen et al. (2000) J. Mol. Recognit. 13:352-359) that mimic the individual and conformational epitopes of the glycoproteins of the invention. Mimitopes may be identified using any technique known in the art, including but not limited to surface stimulation, random peptide libraries or phage display libraries, as well as an antibody or antibodies to the individual and conformational epitopes of the glycoproteins of the invention.

The invention further provides a nucleic acid (e.g., an isolated nucleic acid) encoding a recombinant flavivirus VLP or a recombinant flavivirus particle (e.g., a viral coat of the flavivirus particle) of the invention. In some embodiments, the flavivirus particle or virus like particle (VLP) comprises a recombinant E glycoprotein and/or prM glycoprotein of the present invention.

In some embodiments, a flavivirus particle or VLP of the present invention may comprise a mature form.

Flaviviruses such as dengue virus can adopt either a mature and/or an immature virion structure. As used herein, the term “mature form” refers to the infectious form of a viral particle structure, which in nature is formed from structural changes to the immature form during egress of the virus particle from infected cells. While not wishing to be bound to theory, flaviviruses typically assemble as an immature (e.g., non-infectious) form wherein the surface displayed structural proteins E and prM form trimeric spikes. During egress of the virus particle from the host (infected) cell, the prM protein is cleaved by furin enzyme and the pr component is released from the virus particle upon exposure to neutral pH of the extracellular space. This maturation process allows for structural reconfiguration of the surface proteins that assemble the mature form of a flavivirus virion as a herringbone arrangement of E glycoproteins which lie flat against the surface of the virion (Pierson and Diamond, 2012 Curr. Opin. Virol. 2:168-175).

For example, a mature form of a dengue virus particle comprises a structure of about 90 E glycoprotein homodimers lying flat in a herringbone structure, organized into a 50 nm icosahedral (T=pseudo 3) symmetry. In contrast to the mature form, a dengue virus immature form comprises a structure of a spikey sphere of about 60 nm, with 60 three-fold spikes. A third flavivirus virion form represents partially mature/immature particles which adopt both mature and immature structure in different regions of the particle. Populations of viral particles may comprise mature, immature, and/or partially mature/immature particles, in any combination, in any ratio. Maturation status and affects thereof on viral characteristics are further described in Kuhn et al. 2002 Cell 108:717-725; Zybert et al. 2008 J. Gen. Virol. 89:3047-3051; Plevka et al. 2014 J. Struct. Biol. 185:27-31; Kostyuchenko et al. 2013 J. Virol. 87:7700-7707, Pierson and Diamond, 2012 Curr. Opin. Virol. 2:168-175; and Galula et al. 2019 Hum. Vaccines Immunother. 15:2328-2336, incorporated herein by reference in their entireties.

Accordingly, in some embodiments of the present invention, the mature form of a virus particle comprises wherein the virus particle assembles in a herringbone structure formed from homodimers of E glycoproteins with icosahedral symmetry.

Also provided herein are populations of flavivirus particles of the present invention (e.g., in mature forms, e.g., comprising a recombinant flavivirus prM glycoprotein of the present invention).

In some embodiments, a greater percentage of the flavivirus particles in the population are in a mature form as compared to the percentage of flavivirus particles in a mature form in a population of corresponding wildtype flavivirus particles (e.g., wildtype DV2 particles). For example, in some embodiments, about 10%, 20%, 30%, 40%, 50%, 60%, 70%, 80%, 85%, 90%, 91%, 92%, 93%, 94%, 95%, 96%, 97%, 98%, 99%, or 100% or more or any value or range therein, of the flavivirus particles are in a mature form, as compared to the percentage of flavivirus particles in a mature form in a population of corresponding wildtype flavivirus particles.

The relative amount of mature forms versus immature forms of a population of flavivirus particles may be measured by any standard method in the art, as will be apparent to those skilled in the art in light of the instant disclosure. For example, in some embodiments, the amount of mature forms versus immature forms of a population of flavivirus particles may be measured by determining the ratio of prM glycoprotein versus E glycoprotein (prM/Env) in a population. In some embodiments, the ratio of prM glycoprotein versus E glycoprotein in the population may be less than 1.0 (e.g., 0.99, 0.98, 0.97, 0.96, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.04, 0.03, 0.02, 0.01, or less, or any value or range therein), normalized to the ratio of prM/Env in a population of corresponding wildtype flavivirus particles (e.g., wildtype DV2 particles; e.g., wherein the ratio of prM/Env in the population of the corresponding wildtype flavivirus particles is set to 1.0).

Also provided is a cell (e.g., an isolated cell) comprising a vector, a nucleic acid molecule, a dengue virus protein, a dengue virus peptide, a dengue virus protein domain, a flavivirus protein, a flavivirus peptide, flavivirus protein domain, a dengue virus particle, a dengue virus VLP, a flavivirus VLP and/or a flavivirus particle of this invention, singly or in any combination.

The invention also provides immunogenic compositions comprising the cells, vectors, nucleic acids molecules, dengue virus proteins, dengue virus VLPs, chimeric dengue virus particles, flavivirus VLPs and/or flavivirus particles of the invention, singly or in any combination. In some embodiments, the immunogenic composition is monovalent. In some embodiments, the immunogenic composition is multivalent (e.g., bivalent, trivalent or tetravalent) for flavivirus serotypes, e.g., dengue virus serotypes DENV1, DENV2, DENV3 and/or DENV4 in any combination. The recombinant flavivirus prM glycoproteins of this invention can be administered to a subject singly or in any combination, including any combination of priming and boosting according to such immunization protocols that are known in the art. In some embodiments, a prime/boost combination would be used that results in administration of antigens representative of all four dengue virus serotypes. Such a prime/boost regimen can include administration of any combination of antigens in any order to achieve this result. A nonlimiting example of a prime/boost protocol can include priming at day 0 and boosting at 3 months and 6 months, or boosting at 6 months and 1 year, respectively. This protocol could also be modified to include only one boost at either 3 months, 6 months or 1 year.

The invention also provides immunogenic compositions comprising the cells, vectors, nucleic acid molecules, VLPs, VRPs, dengue virus or flavivirus particles and/or populations of the invention. The composition can further comprise a pharmaceutically acceptable carrier.

By “pharmaceutically acceptable” it is meant a material that is not toxic or otherwise undesirable, i.e., the material may be administered to a subject without causing any undesirable biological effects. For injection, the carrier will typically be a liquid. For other methods of administration (e.g., such as, but not limited to, administration to the mucous membranes of a subject (e.g., via intranasal administration, buccal administration and/or inhalation)), the carrier may be either solid or liquid. For inhalation administration, the carrier will be respirable, and will preferably be in solid or liquid particulate form. The formulations may be conveniently prepared in unit dosage form and may be prepared by any of the methods well known in the art. In some embodiments, that pharmaceutically acceptable carrier can be a sterile solution or composition.

In some embodiments, the present invention provides a pharmaceutical composition comprising a dengue virus or flavivirus E and/or prM glycoprotein, nucleic acid molecule (e.g., an mRNA molecule), vector, VRP, VLP, flavivirus particle, population and/or composition of the present invention, a pharmaceutically acceptable carrier, and, optionally, other medicinal agents, therapeutic agents, pharmaceutical agents, stabilizing agents, buffers, carriers, adjuvants, diluents, etc., which can be included in the composition singly or in any combination and/or ratio.

Immunogenic compositions comprising dengue virus or flavivirus E and/or prM glycoprotein, nucleic acid molecule, vector, VRP, VLP, dengue virus or flavivirus particle, population and/or composition of the present invention may be formulated by any means known in the art. Such compositions, especially vaccines, are typically prepared as injectables, either as liquid solutions or suspensions. Solid forms suitable for solution in, or suspension in, liquid prior to injection may also be prepared. Lyophilized preparations are also suitable. In some embodiments, a pharmaceutical composition of the present invention may be a vaccine formulation, e.g., may comprise dengue virus or flavivirus protein, nucleic acid molecule, vector, VRP, VLP, flavivirus particle, population and/or composition of the present invention and adjuvant(s), optionally in a vaccine diluent. The active immunogenic ingredients are often mixed with excipients and/or carriers that are pharmaceutically acceptable and/or compatible with the active ingredient. Suitable excipients include but are not limited to sterile water, saline, dextrose, glycerol, ethanol, or the like and combinations thereof, as well as stabilizers, e.g., HSA or other suitable proteins and reducing sugars. In addition, if desired, the vaccines or immunogenic compositions may contain minor amounts of auxiliary substances such as wetting and/or emulsifying agents, pH buffering agents, and/or adjuvants that enhance the effectiveness of the vaccine or immunogenic composition.

In some embodiments, a pharmaceutical composition comprising dengue virus or flavivirus E and/or prM glycoprotein, nucleic acid molecule, vector, VRP, VLP, dengue virus or flavivirus particle, population and/or composition of the present invention may further comprise additional agents, such as, but not limited to, additional antigen as part of a cocktail in a vaccine, e.g., a multi-component vaccine wherein the vaccine may additionally include peptides, cells, virus, viral peptides, inactivated virus, etc. Thus, in some embodiments, a pharmaceutical composition comprising a dengue virus or flavivirus E and/or prM glycoprotein, nucleic acid molecule, vector, VRP, VLP, particle, population and/or composition of the present invention, a pharmaceutically acceptable carrier may further comprise additional viral antigen, e.g., a flavivirus antigen in the form of peptides, peptoids, whole flavivirus (e.g., live attenuated and/or inactivated virus), and/or flavivirus-comprising cells (e.g., cells modified to express flaviviral components, e.g., dengue or other flaviviral peptides).

In some embodiments, a pharmaceutical composition comprising a dengue virus or flavivirus E and/or prM glycoprotein, nucleic acid molecule, vector, VRP, VLP, particle, population and/or composition of the present invention, and a pharmaceutically acceptable carrier may further comprise an adjuvant. As used herein, “suitable adjuvant” describes an adjuvant capable of being combined with dengue virus or flavivirus E and/or prM glycoprotein, nucleic acid molecule, vector, VRP, VLP, particle, population and/or composition of this invention to further enhance an immune response without deleterious effect on the subject or the cell of the subject.

The adjuvants of the present invention can be in the form of an amino acid sequence, and/or in the form or a nucleic acid encoding an adjuvant. When in the form of a nucleic acid, the adjuvant can be a component of a nucleic acid encoding the polypeptide(s) or fragment(s) or epitope(s) and/or a separate component of the composition comprising the nucleic acid encoding the polypeptide(s) or fragment(s) or epitope(s) of the invention. According to the present invention, the adjuvant can also be an amino acid sequence that is a peptide, a protein fragment or a whole protein that functions as an adjuvant, and/or the adjuvant can be a nucleic acid encoding a peptide, protein fragment or whole protein that functions as an adjuvant. As used herein, “adjuvant” describes a substance, which can be any immunomodulating substance capable of being combined with a composition of the invention to enhance, improve, or otherwise modulate an immune response in a subject.

In further embodiments, the adjuvant can be, but is not limited to, an immunostimulatory cytokine (including, but not limited to, GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules), SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Suitable adjuvants also include an aluminum salt such as aluminum hydroxide gel (alum), aluminum phosphate, or algannmulin, but may also be a salt of calcium, iron or zinc, or may be an insoluble suspension of acylated tyrosine, or acylated sugars, cationically or anionically derivatized polysaccharides, or polyphosphazenes.

Other adjuvants are well known in the art and include without limitation MF 59, LT-K63, LT-R72 (Pal et al. Vaccine 24(6):766-75 (2005)), QS-21, Freund's adjuvant (complete and incomplete), aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

Additional adjuvants can include, for example, a combination of monophosphoryl lipid A, preferably 3-de-O-acylated monophosphoryl. lipid A (3D-MPL) together with an aluminum salt. An enhanced adjuvant system involves the combination of a monophosphoryl lipid A and a saponin derivative, particularly the combination of QS21 and 3D-MPL as disclosed in PCT publication number WO 94/00153, or a less reactogenic composition where the QS21 is quenched with cholesterol as disclosed in PCT publication number WO 96/33739. A particularly potent adjuvant formulation involving QS21 3D-MPL & tocopherol in an oil in water emulsion is described in PCT publication number WO 95/17210. In addition, the nucleic acid compositions of the invention can include an adjuvant by comprising a nucleotide sequence encoding the antigen and a nucleotide sequence that provides an adjuvant function, such as CpG sequences. Such CpG sequences, or motifs, are well known in the art.

Adjuvants can be combined, either with the compositions of this invention or with other vaccine compositions that can be used in combination with the compositions of this invention.

### Methods of Using

The nucleic acids, proteins, peptides, viruses, vectors, particles, antibodies, VLPs, VRPs, populations, and/or compositions of this invention are intended for use as therapeutic agents and immunological reagents, for example, as antigens, immunogens, vaccines, and/or nucleic acid delivery vehicles. The compositions described herein can be formulated for use as reagents (e.g., to produce antibodies) and/or for administration in a pharmaceutical carrier in accordance with known techniques. See, e.g., Remington, The Science and Practice of Pharmacy (latest edition).

Accordingly, another aspect of the present invention provides a method of producing an immune response to a flavivirus in a subject, comprising administering to the subject an effective amount of a recombinant E and/or prM glycoprotein, flavivirus particle or VLP, nucleic acid molecule, vector, population, and/or composition of the invention, and any combination thereof.

Another aspect of the present invention provides a method of treating a flavivirus infection in a subject, comprising administering to the subject an effective amount of a recombinant E and/or prM glycoprotein, flavivirus particle or VLP, nucleic acid molecule, vector, population, and/or composition of the invention, and any combination thereof.

Another aspect of the present invention provides a method of preventing a disorder associated with flavivirus infection in a subject, comprising administering to the subject an effective amount of a recombinant E and/or prM glycoprotein, flavivirus particle or VLP, nucleic acid molecule, vector, population, and/or composition of the invention, and any combination thereof.

Another aspect of the present invention provides a method of protecting a subject from the effects of a flavivirus infection, comprising administering to the subject an effective amount of a recombinant E and/or prM glycoprotein, flavivirus particle or VLP, nucleic acid molecule, vector, population, and/or composition of the invention, and any combination thereof.

By “protecting a subject from the effects of a flavivirus infection” it is meant that the subject does not develop a disease or disorder caused by a flavivirus infection, or if the subject does develop a disease or disorder caused by a flavivirus infection, the disease or disorder is of less severity and/or symptoms are reduced and/or less severe in the subject in comparison to what the subject would experience upon infection by a flavivirus in the absence of the administration of the flavivirus protein, a flavivirus protein domain, a flavivirus peptide, a flavivirus particle, a flavivirus VLP, a nucleic acid molecule, a vector, a cell, and/or immunogenic composition of this invention.

Another aspect of the present invention provides a method of identifying the presence of a neutralizing antibody to a flavivirus in a biological sample from a subject, comprising: a) administering a composition comprising an E glycoprotein of the present invention to the subject in an amount effective to induce an antibody response to the E glycoprotein; b) contacting a biological sample from the subject with flavivirus particles comprising the E glycoprotein of step (a) above under conditions whereby neutralization of the flavivirus particles can be detected; and c) detecting neutralization in step (b), thereby identifying the presence of a neutralizing antibody to the flavivirus in the biological sample from the subject.

Another aspect of the present invention provides a method of identifying the presence of a neutralizing antibody to a flavivirus in a biological sample from a subject, comprising: a) contacting a biological sample from a subject that has been administered an E glycoprotein of the present invention with flavivirus particles comprising the E glycoprotein under conditions whereby neutralization of the flavivirus particles can be detected; and b) detecting neutralization in step (a), thereby identifying the presence of a neutralizing antibody to the flavivirus in the biological sample from the subject.

Another aspect of the present invention provides a method of identifying an immunogenic composition that induces a neutralizing antibody to a flavivirus in a subject, the method comprising: a) administering an immunogenic composition comprising an E glycoprotein of the present invention to a subject in an amount effective to induce an antibody response to the E glycoprotein; b) contacting a biological sample from the subject with flavivirus particles comprising the E glycoprotein of step (a) under conditions whereby neutralization of the flavivirus particles can be detected; c) determining if the biological sample comprises an antibody that neutralizes flavivirus particles comprising the E glycoprotein of step (a); and d) identifying the immunogenic composition as inducing a neutralizing antibody to the flavivirus in the subject if the biological sample comprises an antibody that neutralizes flavivirus particles comprising the E glycoprotein of (a).

Another aspect of the present invention provides a method of identifying an immunogenic composition that induces a neutralizing antibody to a flavivirus in a subject, the method comprising: a) contacting a biological sample from a subject that has been administered an immunogenic composition comprising an E glycoprotein of the present invention with flavivirus particles comprising the E glycoprotein under conditions whereby neutralization of the flavivirus particles can be detected; b) determining if the biological sample comprises an antibody that neutralizes flavivirus particles comprising the E glycoprotein of step (a); and c) identifying the immunogenic composition as inducing a neutralizing antibody to the flavivirus in the subject if the biological sample comprises an antibody that neutralizes flavivirus particles comprising the E glycoprotein of (a).

Another aspect of the present invention provides a method of method of detecting an antibody to a flavivirus in a sample, comprising: a) contacting the sample with an E glycoprotein of the present invention under conditions whereby an antigen/antibody complex can form; and b) detecting formation of an antigen/antibody complex, thereby detecting an antibody to the flavivirus in the sample.

Another aspect of the present invention provides a method of identifying an antibody to a flavivirus in a biological sample from a subject, comprising: a) administering a composition comprising an E glycoprotein of the present invention to the subject in an amount effective to induce an antibody response to the E glycoprotein; b) contacting a biological sample from the subject with the E glycoprotein of (a) under conditions whereby an antigen/antibody complex can form; and c) detecting formation of an antigen/antibody complex, thereby identifying an antibody to the flavivirus in the biological sample from the subject.

Another aspect of the present invention provides a method of identifying an antibody to a flavivirus in a biological sample from a subject, comprising: a) contacting a biological sample from a subject that has been administered an immunogenic composition comprising an E glycoprotein of the present invention with the E glycoprotein under conditions whereby an antigen/antibody complex can form; and b) detecting formation of an antigen/antibody complex, thereby identifying an antibody to the flavivirus in the biological sample from the subject.

Another aspect of the present invention provides a method of identifying an immunogenic composition that induces an antibody to a flavivirus in a subject, the method comprising: a) contacting a biological sample from a subject that has been administered an immunogenic composition comprising an E glycoprotein of the present invention with the E glycoprotein under conditions whereby an antigen/antibody complex can form; and detecting formation of an antigen/antibody complex, thereby identifying an immunogenic composition that induces an antibody to the flavivirus in the subject.

Another aspect of the present invention provides a method of identifying an immunogenic composition that induces a neutralizing antibody to a flavivirus in a subject, comprising: a) administering an immunogenic composition comprising an E glycoprotein of the present invention to a subject in an amount effective to induce an antibody response to the E glycoprotein; b) contacting a biological sample from the subject with the E glycoprotein of (a) under conditions whereby an antigen/antibody complex can form; and c) detecting formation an antigen/antibody complex, thereby identifying an immunogenic composition that induces a neutralizing antibody to the flavivirus in the subject.

In some embodiments, the antibody (e.g., the neutralizing antibody, the antibody of the antigen/antibody complex), produced binds to the E glycoprotein fusion loop epitope.

In some embodiments, the antibody (e.g., the neutralizing antibody, the antibody of the antigen/antibody complex), produced is an antibody-dependent enhancement (ADE) antibody (e.g., an antibody associated with an ADE response).

In some embodiments, the antibody (e.g., the neutralizing antibody, the antibody of the antigen/antibody complex), produced is not an antibody-dependent enhancement (ADE) antibody (e.g., not an antibody associated with an ADE response).

Regarding dengue viruses, there are four serotypes of dengue virus (DENV-1, DENV-2, DENV-3 and DENV-4). Within each serotype there are a number of different strains or genotypes. The dengue virus antigens and epitopes of the invention can be derived from any dengue virus, including all serotypes, strains and genotypes, now known or later identified.

In some embodiments of the invention, the dengue virus may be UNC1017 strain (DENV1), West Pacific 74 strain (DENV1), S16803 strain (DENV2), UNC2005 strain (DENV2), UNC3001 strain (DENV3), UNC3043 (DENV3 strain 059.AP-2 from Philippines, 1984), UNC3009 strain (DENV3, D2863, Sri Lanka 1989), UNC3066 (DENV3, strain 1342 from Puerto Rico 1977), CH53489 strain (DENV3), Indonesia 1982 (DENV3), Cuba 2002 (DENV3), UNC4019 strain (DENV4), or TVP-360 (DENV4).

The present invention provides additional non limiting examples of recombinant flavivirus E and/or prM glycoproteins of this invention that can be used in the compositions and methods described herein in the SEQUENCES section provided herein.

In some embodiments of the methods of the present invention, the method may further comprise co-administering and/or co-contacting with a modified prM glycoprotein. In some embodiments, the method may further comprise co-administering and/or co-contacting with a modified prM glycoprotein of the present invention.

In some embodiments, the co-administered and/or co-contacted modified prM glycoprotein is: encoded on a/the administered and/or contacted nucleic acid molecule comprising the E glycoprotein (i.e., a nucleic acid molecule encoding the modified prM glycoprotein and the E glycoprotein); comprised within a/the administered and/or contacted flavivirus particle or VLP comprising the E glycoprotein (i.e., a flavivirus particle or VLP comprising the modified prM glycoprotein and the E glycoprotein); comprised within a/the administered and/or contacted vector comprising the E glycoprotein (i.e., a vector comprising the modified prM glycoprotein and the E glycoprotein); and/or comprised within a/the administered and/or contacted population comprising the E glycoprotein (i.e., a population of flavivirus particles or VLP comprising the modified prM glycoprotein and the E glycoprotein, e.g., a mixed population of one or more flavivirus particle or VLP comprising the E glycoprotein and one or more flavivirus particle or VLP comprising the modified prM protein and/or a population of flavivirus particles or VLP comprising one or more flavivirus particle comprising the modified prM protein and the E glycoprotein).

In some embodiments of the methods of the present invention, the particle is in a mature form.

In some embodiments of the methods disclosed herein, the sample is a biological sample from a subject.

The present invention can be practiced for prophylactic, therapeutic and/or diagnostic purposes. In addition, the invention can be practiced to produce antibodies for any purpose, such as diagnostic or research purposes, or for passive immunization by transfer to another subject.

For example, the recombinant E and/or prM proteins and/or flavivirus particles comprising the same of this invention can be used to immunize a subject against infection by a new flavivirus, as well as treat a subject infected with a newly emerging flavivirus.

The recombinant flavivirus E and/or prM glycoprotein of the present invention may be administered in any frequency, amount, and/or route as needed to elicit an effective prophylactic and/or therapeutic effect in a subject (e.g., in a subject in need thereof) as described herein. In certain embodiments, the recombinant flavivirus E and/or prM glycoprotein, nucleic acid molecule, vector, VRP, VLP, flavivirus particle, population and/or composition is administered/delivered to the subject, e.g., systemically (e.g., intravenously). In particular embodiments, more than one administration (e.g., two, three, four or more administrations) may be employed to achieve the desired level of protein expression over a period of various intervals, e.g., daily, weekly, monthly, yearly, etc. The most suitable route in any given case will depend on the nature and severity of the condition being treated and on the nature of the particular delivery method that is being used. In embodiments wherein a vector is used, the vector will typically be administered in a liquid formulation by direct injection (e.g., stereotactic injection) to the desired region or tissues. In some embodiments, the vector can be delivered via a reservoir and/or pump. In other embodiments, the vector may be provided by topical application to the desired region or by intra-nasal administration of an aerosol formulation. Administration to the eye or into the ear, may be by topical application of liquid droplets. As a further alternative, the vector may be administered as a solid, slow-release formulation. For example, controlled release of parvovirus and AAV vectors is described in international patent publication WO 01/91803, which is incorporated by reference herein for these teachings.

Administration may be by any suitable means, such as intraperitoneally, intramuscularly, intranasally, intravenously, intradermally (e.g., by a gene gun), intrarectally and/or subcutaneously. The compositions herein may be administered via a skin scarification method, and/or transdermally via a patch or liquid. The compositions can be delivered subdermally in the form of a biodegradable material that releases the compositions over a period of time. As further non-limiting examples, the route of administration can be by inhalation (e.g., oral and/or nasal inhalation), oral, buccal (e.g., sublingual), rectal, vaginal, topical (including administration to the airways), intraocular, by parenteral (e.g., intramuscular [e.g., administration to skeletal muscle], intravenous, intra-arterial, intraperitoneal and the like), subcutaneous (including administration into the footpad), intrapleural, intracerebral, intrathecal, intraventricular, intra-aural, intra-ocular (e.g., intra-vitreous, sub-retinal, anterior chamber) and peri-ocular (e.g., sub-Tenon's region) routes or any combination thereof.

In some embodiments, recombinant E and/or prM proteins and/or flavivirus particles comprising the same can be administered to a subject as a nucleic acid molecule, which can be a naked nucleic acid molecule or a nucleic acid molecule present in a vector (e.g., a delivery vector, which in some embodiments can be a viral vector, such as a VRP). The nucleic acids and vectors of this invention can be administered orally, intranasally, parenterally (e.g., intravenously), by intramuscular injection, by intraperitoneal injection, transdermally, extracorporeally, topically or the like. In the methods described herein which include the administration and uptake of exogenous DNA into the cells of a subject (i.e., gene transduction or transfection), the nucleic acids of the present invention can be in the form of naked DNA or the nucleic acids can be in a vector for delivering the nucleic acids to the cells for expression of the polypeptides and/or fragments of this invention. The vector can be a commercially available preparation or can be constructed in the laboratory according to methods well known in the art.

Delivery of the nucleic acid or vector to cells can be via a variety of mechanisms, including but not limited to recombinant vectors including bacterial, viral, and fungal vectors, liposomal delivery agents, nanoparticles, and gene gun related mechanisms.

In some embodiments, the nucleic acid molecules encoding recombinant flavivirus E and/or prM glycoprotein of this invention can be part of a recombinant nucleic acid construct comprising any combination of restriction sites and/or functional elements as are well known in the art that facilitate molecular cloning and other recombinant nucleic acid manipulations. Thus, the present invention further provides a recombinant nucleic acid construct comprising a nucleic acid molecule encoding recombinant flavivirus E and/or prM glycoprotein of this invention. The nucleic acid molecule encoding recombinant flavivirus E and/or prM glycoprotein of this invention can be any nucleic acid molecule that functionally encodes the recombinant flavivirus E and/or prM glycoprotein of this invention. To functionally encode recombinant flavivirus E and/or prM glycoprotein (i.e., allow the nucleic acids to be expressed), the nucleic acid of this invention can include, for example, expression control sequences, such as an origin of replication, a promoter, an enhancer and necessary information processing sites, such as ribosome binding sites, RNA splice sites, polyadenylation sites and transcriptional terminator sequences.

Non-limiting examples of expression control sequences that can be present in a nucleic acid molecule of this invention include promoters derived from metallothionine genes, actin genes, immunoglobulin genes, CMV, SV40, adenovirus, bovine papilloma virus, etc. A nucleic acid molecule encoding a selected recombinant prM glycoprotein can readily be determined based upon the genetic code for the amino acid sequence of the selected polypeptide and/or fragment of interest included in the recombinant prM glycoprotein, and many nucleic acids will encode any selected polypeptide and/or fragment. Modifications in the nucleic acid sequence encoding the polypeptide and/or fragment are also contemplated. Modifications that can be useful are modifications to the sequences controlling expression of the polypeptide and/or fragment to make production of the polypeptide and/or fragment inducible or repressible as controlled by the appropriate inducer or repressor. Such methods are standard in the art. The nucleic acid molecule and/or vector of this invention can be generated by means standard in the art, such as by recombinant nucleic acid techniques and/or by synthetic nucleic acid synthesis or in vitro enzymatic synthesis.

The nucleic acids and/or vectors of this invention can be transferred into a host cell (e.g., a prokaryotic or eukaryotic cell) by well-known methods, which vary depending on the type of cell host. For example, calcium chloride transfection is commonly used for prokaryotic cells, whereas calcium phosphate treatment, transduction, cationic lipid treatment and/or electroporation can be used for other cell hosts.

As another example, delivery can be via a liposome, using commercially available liposome preparations such as LIPOFECTIN, LIPOFECTAMINE (GIBCO-BRL, Inc., Gaithersburg, MD), SUPERFECT (Qiagen, Inc. Hilden, Germany) and TRANSFECTAM (Promega, Madison, WI), as well as other liposomes developed according to procedures standard in the art. In addition, the nucleic acid or vector of this invention can be delivered in vivo by electroporation, the technology for which is available from Genetronics, Inc. (San Diego, CA) as well as by means of a SONOPORATION machine (ImaRx Pharmaceutical Corp., Tucson, AZ).

As another example, vector delivery can be via a viral system, such as a retroviral vector system, which can package a recombinant retroviral genome. The recombinant retrovirus can then be used to infect and thereby deliver to the infected cells nucleic acid encoding the polypeptide and/or fragment of this invention. The exact method of introducing the exogenous nucleic acid into mammalian cells is, of course, not limited to the use of retroviral vectors. Other techniques are widely available for this procedure including the use of adenoviral vectors, alphaviral vectors (e.g., VRPs), adeno-associated viral (AAV) vectors, lentiviral vectors, pseudotyped retroviral vectors and vaccinia viral vectors, as well as any other viral vectors now known or developed in the future. Physical transduction techniques can also be used, such as liposome delivery and receptor-mediated and other endocytosis mechanisms. This invention can be used in conjunction with any of these or other commonly used gene transfer methods.

### Methods of Making

Another aspect of the present invention provides a method of producing a flavivirus particle, comprising: constructing a flavivirus particle comprising the recombinant E glycoprotein of the present invention; thereby producing a flavivirus particle comprising the recombinant E glycoprotein.

In some embodiments, the method may further comprise introducing into the flavivirus particle a modified prM glycoprotein (e.g., the modified prM glycoprotein of the present invention). In some embodiments, the presence of the modified prM glycoprotein forms the flavivirus particle in a mature form (e.g., assembles the particle in a herringbone structure formed from homodimers of E glycoproteins with icosahedral symmetry).

Another aspect of the present invention provides a method of producing a mature flavivirus particle, comprising: constructing a flavivirus particle comprising a recombinant E glycoprotein of the present invention and a modified prM glycoprotein (e.g., (e.g., the modified prM glycoprotein of the present invention), wherein the presence of the modified prM glycoprotein induces the flavivirus particle to assemble in a mature form (e.g., assemble in a herringbone structure formed from homodimers of E glycoproteins with icosahedral symmetry); thereby producing a mature flavivirus particle.

In some embodiments of the methods disclosed herein, the flavivirus particle may comprise a population of flavivirus particles.

The recombinant E and/or prM glycoproteins of the present invention may be substituted by any standard known or later developed method for mutagenesis, as would be understood by the skilled artisan upon review of the disclosures herein. Non-limiting examples of mutagenesis methods include, but are limited to, site-directed mutagenesis, saturation mutagenesis, directed evolution, deep mutagenesis, unnatural amino acid incorporation, post-translational modification, or any combination thereof.

### Kits and Pharmaceutical Formulations

The present invention further provides a kit comprising one or more compositions of this invention. It would be well understood by one of ordinary skill in the art that the kit of this invention can comprise one or more containers and/or receptacles to hold the reagents (e.g., antibodies, antigens, nucleic acids) of the kit, along with appropriate buffers and/or diluents and/or other solutions and directions for using the kit, as would be well known in the art. Such kits can further comprise adjuvants and/or other immunostimulatory or immunomodulating agents, as are well known in the art.

The compositions and kits of the present invention can also include other medicinal agents, pharmaceutical agents, carriers, diluents, immunostimulatory cytokines, etc. Actual methods of preparing such dosage forms are known, or will be apparent, to those skilled in this art.

Immunomodulatory compounds, such as immunomodulatory chemokines and cytokines (preferably, CTL inductive cytokines) can be administered concurrently to a subject.

Cytokines may be administered by any method known in the art. Exogenous cytokines may be administered to the subject, or alternatively, a nucleic acid encoding a cytokine may be delivered to the subject using a suitable vector, and the cytokine produced in vivo. In particular embodiments, a viral adjuvant expresses the cytokine.

In embodiments of the invention, multiple dosages (e.g., two, three or more) of a composition of the invention can be administered without detectable pathogenicity (e.g., Dengue Shock Syndrome/Dengue Hemorrhagic Fever).

If ex vivo methods are employed, cells or tissues can be removed and maintained outside the body according to standard protocols well known in the art. The nucleic acids and vectors of this invention can be introduced into the cells via any gene transfer mechanism, such as, for example, virus-mediated gene delivery, calcium phosphate mediated gene delivery, electroporation, microinjection or proteoliposomes. The transduced cells can then be infused (e.g., in a pharmaceutically acceptable carrier) or transplanted back into the subject per standard methods for the cell or tissue type. Standard methods are known for transplantation or infusion of various cells into a subject.

In embodiments of the invention, the multivalent vaccines of the invention do not result in immune interference, e.g., a balanced immune response is induced against all antigens presented. In embodiments of the invention, the balanced response results in protective immunity against DENV-1, DENV-2, DENV-3 and DENV-4.

In embodiments of the invention, the multivalent vaccine can be administered to a subject that has anti-dengue maternal antibodies present.

An adjuvant for use with the present invention, such as any adjuvant disclosed herein, for example, an immunostimulatory cytokine, can be administered before, concurrent with, and/or within a few hours, several hours, and/or 1, 2, 3, 4, 5, 6, 7, 8, 9, and/or 10 days before and/or after the administration of a composition of the invention to a subject.

Furthermore, any combination of adjuvants, such as immunostimulatory cytokines, can be co-administered to the subject before, after and/or concurrent with the administration of an immunogenic composition of the invention. For example, combinations of immunostimulatory cytokines, can consist of two or more immunostimulatory cytokines, such as GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules. The effectiveness of an adjuvant or combination of adjuvants can be determined by measuring the immune response produced in response to administration of a composition of this invention to a subject with and without the adjuvant or combination of adjuvants, using standard procedures, as described herein and as known in the art.

In embodiments of the invention, the adjuvant comprises an alphavirus adjuvant as described, for example in U.S. Pat. No. 7,862,829.

Boosting dosages can further be administered over a time course of days, weeks, months or years. In chronic infection, initial high doses followed by boosting doses may be advantageous.

The pharmaceutical formulations of the invention can optionally comprise other medicinal agents, pharmaceutical agents, stabilizing agents, buffers, carriers, diluents, salts, tonicity adjusting agents, wetting agents, and the like, for example, sodium acetate, sodium lactate, sodium chloride, potassium chloride, calcium chloride, sorbitan monolaurate, triethanolamine oleate, etc.

For injection, the carrier will typically be a liquid. For other methods of administration, the carrier may be either solid or liquid. For inhalation administration, the carrier will be respirable, and is typically in a solid or liquid particulate form.

The compositions of the invention can be formulated for administration in a pharmaceutical carrier in accordance with known techniques. See, e.g., Remington, The Science And Practice of Pharmacy (9^{th} Ed. 1995). In the manufacture of a pharmaceutical composition according to the invention, the VLPs are typically admixed with, inter alia, an acceptable carrier. The carrier can be a solid or a liquid, or both, and is optionally formulated with the compound as a unit-dose formulation, for example, a tablet. A variety of pharmaceutically acceptable aqueous carriers can be used, e.g., water, buffered water, 0.9% saline, 0.3% glycine, hyaluronic acid, pyrogen-free water, pyrogen-free phosphate-buffered saline solution, bacteriostatic water, or Cremophor EL[R] (BASF, Parsippany, N.J.), and the like. These compositions can be sterilized by conventional techniques. The formulations of the invention can be prepared by any of the well-known techniques of pharmacy.

The pharmaceutical formulations can be packaged for use as is, or lyophilized, the lyophilized preparation generally being combined with a sterile aqueous solution prior to administration. The compositions can further be packaged in unit/dose or multi-dose containers, for example, in sealed ampoules and vials.

The pharmaceutical formulations can be formulated for administration by any method known in the art according to conventional techniques of pharmacy. For example, the compositions can be formulated to be administered intranasally, by inhalation (e.g., oral inhalation), orally, buccally (e.g., sublingually), rectally, vaginally, topically, intrathecally, intraocularly, transdermally, by parenteral administration (e.g., intramuscular [e.g., skeletal muscle], intravenous, subcutaneous, intradermal, intrapleural, intracerebral and intra-arterial, intrathecal), or topically (e.g., to both skin and mucosal surfaces, including airway surfaces).

For intranasal or inhalation administration, the pharmaceutical formulation can be formulated as an aerosol (this term including both liquid and dry powder aerosols). For example, the pharmaceutical formulation can be provided in a finely divided form along with a surfactant and propellant. Typical percentages of the composition are 0.01-20% by weight, preferably 1-10%. The surfactant is generally nontoxic and soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1-20% by weight of the composition, preferably 0.25-5%. The balance of the composition is ordinarily propellant. A carrier can also be included, if desired, as with lecithin for intranasal delivery. Aerosols of liquid particles can be produced by any suitable means, such as with a pressure-driven aerosol nebulizer or an ultrasonic nebulizer, as is known to those of skill in the art. See, e.g., U.S. Pat. No. 4,501,729. Aerosols of solid particles can likewise be produced with any solid particulate medicament aerosol generator, by techniques known in the pharmaceutical art. Intranasal administration can also be by droplet administration to a nasal surface.

Injectable formulations can be prepared in conventional forms, either as liquid solutions or suspensions, solid forms suitable for solution or suspension in liquid prior to injection, or as emulsions. Alternatively, one can administer the pharmaceutical formulations in a local rather than systemic manner, for example, in a depot or sustained-release formulation.

Extemporaneous injection solutions and suspensions can be prepared from sterile powders, granules and tablets of the kind previously described. For example, an injectable, stable, sterile formulation of the invention in a unit dosage form in a sealed container can be provided. The formulation can be provided in the form of a lyophilizate, which can be reconstituted with a suitable pharmaceutically acceptable carrier to form a liquid composition suitable for injection into a subject. The unit dosage form can be from about 1 μg to about 10 grams of the formulation. When the formulation is substantially water-insoluble, a sufficient amount of emulsifying agent, which is pharmaceutically acceptable, can be included in sufficient quantity to emulsify the formulation in an aqueous carrier. One such useful emulsifying agent is phosphatidyl choline.

Pharmaceutical formulations suitable for oral administration can be presented in discrete units, such as capsules, cachets, lozenges, or tables, as a powder or granules; as a solution or a suspension in an aqueous or non-aqueous liquid; or as an oil-in-water or water-in-oil emulsion. Oral delivery can be performed by complexing a compound(s) of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the protein(s) and a suitable carrier (which may contain one or more accessory ingredients as noted above). In general, the pharmaceutical formulations are prepared by uniformly and intimately admixing the compound(s) with a liquid or finely divided solid carrier, or both, and then, if necessary, shaping the resulting mixture. For example, a tablet can be prepared by compressing or molding a powder or granules, optionally with one or more accessory ingredients. Compressed tablets are prepared by compressing, in a suitable machine, the formulation in a free-flowing form, such as a powder or granules optionally mixed with a binder, lubricant, inert diluent, and/or surface active/dispersing agent(s). Molded tablets are made by molding, in a suitable machine, the powdered protein moistened with an inert liquid binder.

Pharmaceutical formulations suitable for buccal (sub-lingual) administration include lozenges comprising the compound(s) in a flavored base, usually sucrose and acacia or tragacanth; and pastilles in an inert base such as gelatin and glycerin or sucrose and acacia.

Pharmaceutical formulations suitable for parenteral administration can comprise sterile aqueous and non-aqueous injection solutions, which preparations are preferably isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of nonaqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical formulations suitable for rectal administration are optionally presented as unit dose suppositories. These can be prepared by admixing the active agent with one or more conventional solid carriers, such as for example, cocoa butter and then shaping the resulting mixture.

Pharmaceutical formulations suitable for topical application to the skin preferably take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical formulation of the present invention with a lipophilic reagent (e.g., DMSO) that is capable of passing into the skin.

Pharmaceutical formulations suitable for transdermal administration can be in the form of discrete patches adapted to remain in intimate contact with the epidermis of the subject for a prolonged period of time. Formulations suitable for transdermal administration can also be delivered by iontophoresis (see, for example, Pharmaceutical Research 3:318 (1986)) and typically take the form of a buffered aqueous solution of the compound(s). Suitable formulations can comprise citrate or bis\tris buffer (pH 6) or ethanol/water and can contain from 0.1 to 0.2M active ingredient.

In embodiments of the invention, the dosage of a virus particle of this invention can be in a range of about 10⁴ to about 10⁷ plaque forming units (PFUs). In embodiments of this invention, the dosage of a VLP of this invention can be in a range of about 500 micrograms to about 5 milligrams. In embodiments of this invention, the dosage of a protein of this invention can be in a range of about 10⁰ to about 10⁴ micrograms+/−adjuvant.

Further, the composition can be formulated as a liposomal formulation. The lipid layer employed can be of any conventional composition and can either contain cholesterol or can be cholesterol-free. The liposomes that are produced can be reduced in size, for example, through the use of standard sonication and homogenization techniques.

The liposomal formulations can be lyophilized to produce a lyophilizate which can be reconstituted with a pharmaceutically acceptable carrier, such as water, to regenerate a liposomal suspension.

The immunogenic formulations of the invention can optionally be sterile, and can further be provided in a closed pathogen-impermeable container.

The present subject matter will now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### EXAMPLES

The following examples provide illustrative embodiments. Certain aspects of the following examples are disclosed in terms of techniques and procedures found or contemplated by the present inventors to work well in the practice of the embodiments. In light of the present disclosure and the general level of skill in the art, those of skill will appreciate that the following examples are intended to be exemplary only and that numerous changes, modifications, and alterations can be employed without departing from the scope of the presently claimed subject matter.

### Example 1

To evolve a novel fusion loop epitope, we generated two different saturation mutagenesis libraries, each with 5 randomized amino acids: DRGXGXGXXXFGK (Library 1; SEQ ID NO:13) and DRGXXXXXGLFGK (Library 2; SEQ ID NO:14). Library 1 was designed to mutate known important residues for FL-Ab binding while Library 2 focused on a continuous linear epitope to maximally alter antigenicity (Smith et al., 2013 mBio 4.6: e00873-13). These plasmid libraries were used to produce viral libraries in either C6/36 (Aedes albopictus mosquito) or Vero 81 (African green monkey) cells and passaged three times in their respective cell types. Following the directed evolution process, viral genomes were extracted and subjected to deep sequencing to identify surviving and enriched variants (FIG. 1 panel B). Due to the high level of conservation, it was unsurprising that not all libraries and conditions yielded viable progeny; evolutions carried out on Library 2 only yielded wild-type sequences. For Library 1, primarily wild-type sequences were recovered from Vero 81 evolved libraries. However, when we evolved Library 1 in C6/36 cells, a novel variant was selected with two amino acid changes: DRGWGSGCLLFGK (SEQ ID NO:10). This variant was the only major sequence found, comprising ˜25% of the population, while the next most populous variant comprised 0.06% of the population (FIG. 1 panel C). Residues W101, C105, and L107 preserved in our final sequence, supporting the structural importance of these residues (Modis et al. 2004 Nature 427:313-319). When modeled on the DENV2 structure, the N103S and G106L mutations are located at the interface with the neighboring monomer EDIII domain (FIG. 1 panel D). Using this sequence, we created an infectious clone, DENV2-FL, containing the identified amino acid changes. We also created DENV2-FL-Mature (DENV2-FL-M; FIG. 1 panel E), containing both the evolved fusion loop motif and an evolved prM furin cleavage site (Tse et al. 2022 mBio 13:1-18).

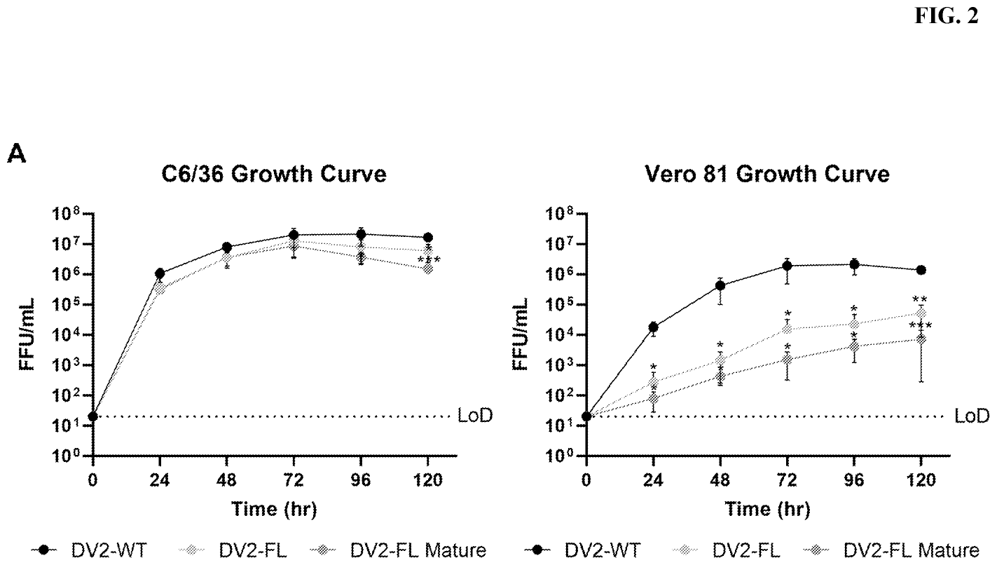

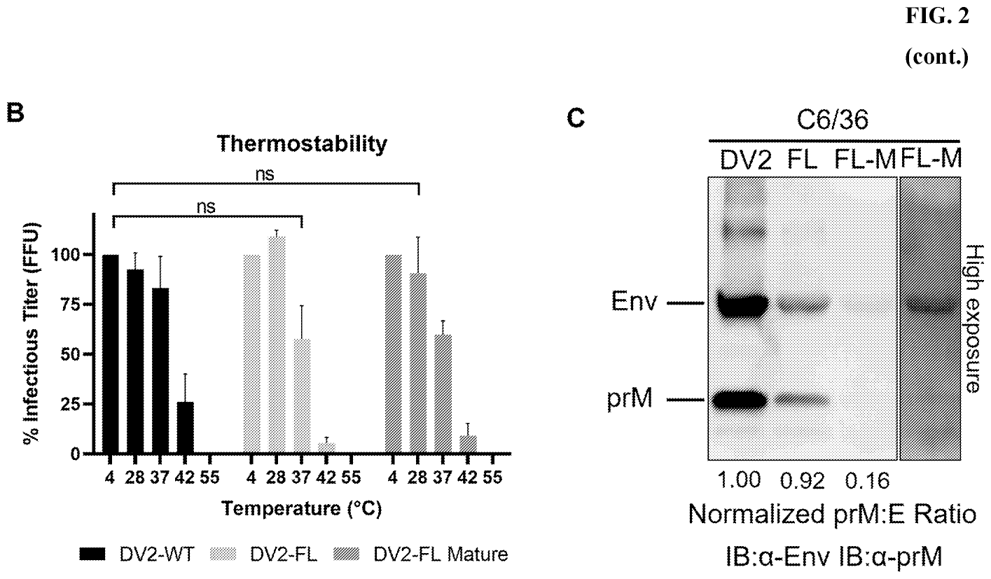

We performed growth curves comparing DENV2, DENV2-FL, and DENV2-FL-Mature in both C6/36 and Vero 81 cells. In C6/36 cells, growth of all three viruses was comparable, reaching high 10⁶-10⁷ titers. However, in Vero 81 cells, both fusion loop mutant viruses were highly attenuated, with a multiple-log drop in titer (FIG. 2 panel A). The change in cell line involves both a change from insect to mammalian cells, as well as a change in growth temperature. To investigate the role of temperature change, we performed a thermostability assay, comparing virus incubated at temperatures ranging from 28-55° C. before infection. The three viruses had comparable thermostabilities, indicating that this does not explain the attenuation of the fusion loop mutants (FIG. 2 panel B). Because the DENV2-FL-Mature virus contains a mutation to increase prM cleavage, we also assayed virion maturation of the three viruses. DENV2-FL had a comparable prM:E ratio to DENV2, while DENV2-FL-Mature had a reduced prM:E ratio, indicating a higher degree of maturation (FIG. 2 panel C). As the DENV2-FL-Mature virus contains both evolved motifs, we focused on this variant for further study.

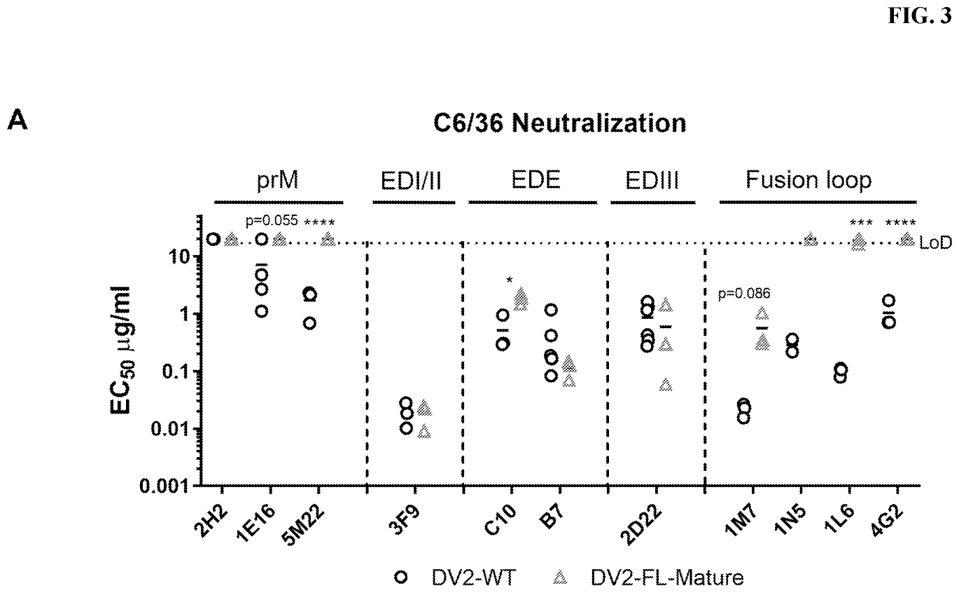

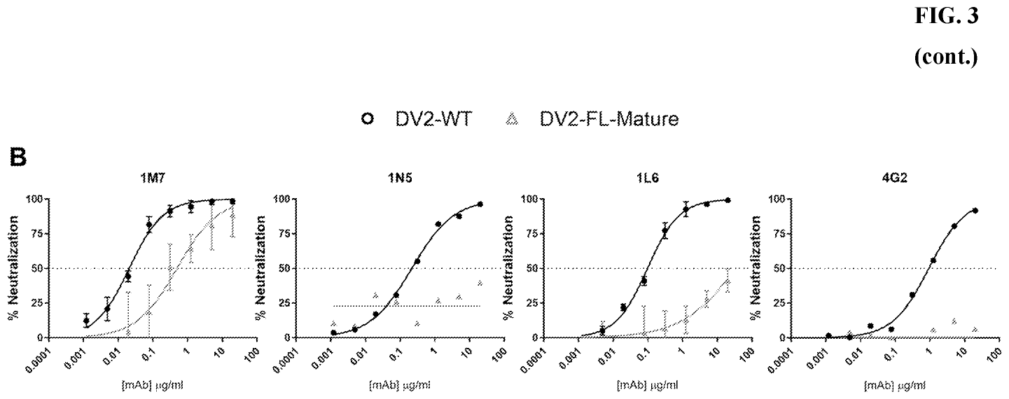

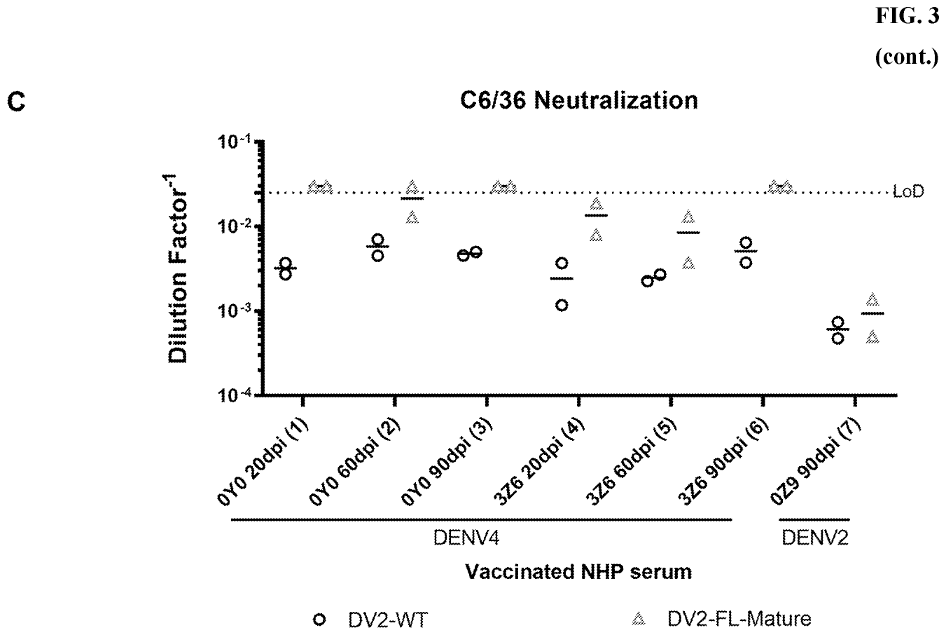

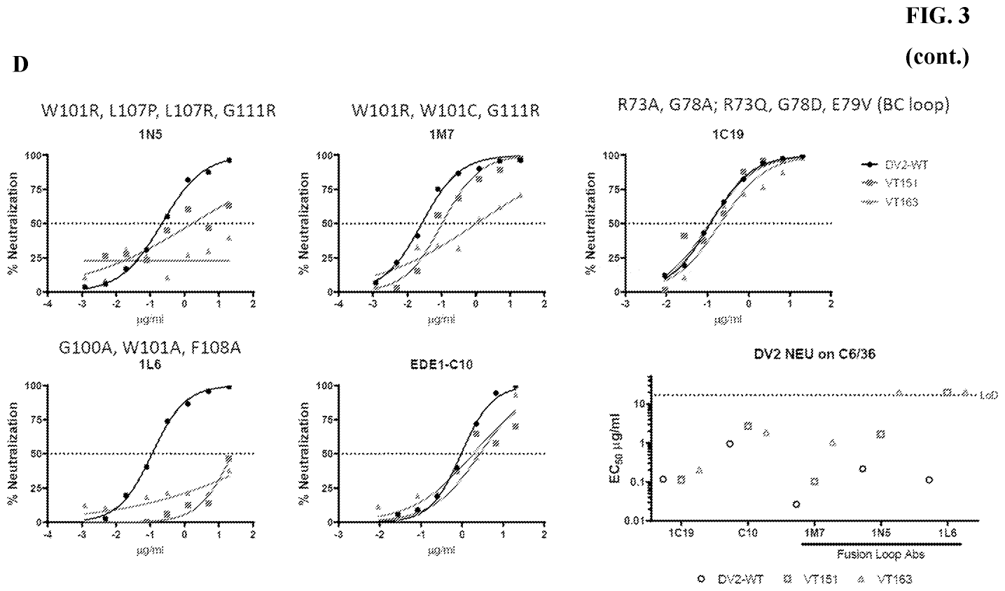

We characterized the antigenicity of DENV2-FL-Mature with a panel of monoclonal Abs. Virus containing the evolved furin cleavage site is unable to be neutralized by the prM Abs 1E16 and 5M22. For Abs targeting unaffected epitopes, including EDI/II, EDE, and EDIII, EC₅₀ values were generally comparable between DENV2 and DENV2-FL-Mature, indicating overall virion structural integrity (FIG. 3 panel A). However, DENV2-FL-Mature is insensitive to Abs targeting the FL. Neutralization by 1M7 is reduced by ˜2-logs, and no neutralization was observed for 1N5, 1L6, and 4G2 (FIG. 3 panels A, B, D).

Finally, we analyzed neutralization of the DENV2-FL-Mature virus in sera derived from immunized non-human primates (NHPs). Control serum from a NHP vaccinated with DENV2 did not display a difference in neutralization between wildtype DENV2 and DENV2-FL-Mature, confirming that prM or fusion loop epitopes are not major contributors to the homotypic type-specific (TS) Ab response. However, in NHPs vaccinated with DENV4, no heterologous protection is observed against the DENV2-FL-Mature virus while strong neutralization potency was demonstrated against wildtype DENV2. One animal (3Z6) showed low levels of neutralization at early time points (20- and 60-days post vaccination) which was eventually lost at later time points. This may indicate that after a single dose of vaccination, much of the cross-reactive (CR) Ab responses target prM and the fusion loop while other protective CR antibodies wane overtime (FIG. 3 panels C and D).

In this study we engineered a viable variant of DENV2 containing an evolved FL motif, which due to complete conservation was previously believed to be unchangeable while retaining function. We further developed a double mutant variant containing the novel FL motif and our evolved prM furin cleavage site for increased maturation. This newly evolved virus is insensitive to FL and prM Abs while still neutralized by the important protective TS and CR EDE Abs. DENV2-FL-Mature can discriminate the ADE-prone FL- and prM-Abs from the truly protective CR Abs in polyclonal serum, better reflecting in vivo protection.

### Example 2: Methods Related to Example 1

Cells and viruses: C6/36 (ATCC CRL-1660) were grown in MEM (Gibco) with 5% FBS (HyClone), 1% penicillin/streptomycin (Gibco), 0.1 mM nonessential amino acids (Gibco), 1% HEPES (Gibco), and 2 mM GlutaMAX (Gibco), cultured at 32° C. with 5% CO₂. Vero 81 cells (ATCC CCL-81) were grown in DMEM/F12 (Gibco) with 10% FBS, 1% penicillin/streptomycin (Gibco), 0.1 mM nonessential amino acids (Gibco), and 1% HEPES (Gibco), cultured at 37° C. with 5% CO₂. DENV viruses were grown in C6/36 or Vero 81 cells maintained in infection media. C6/36 infection media consists of Opti-MEM (Gibco) with 2% FBS (HyClone), 1% penicillin/streptomycin (Gibco), 0.1 mM nonessential amino acids (Gibco), 1% HEPES (Gibco), and 2 mM GlutaMAX (Gibco). Vero 81 infection media consists of DMEM/F12 (Gibco) with 2% FBS, 1% penicillin/streptomycin (Gibco), 0.1 mM nonessential amino acids (Gibco), and 1% HEPES (Gibco). DENV2 strain S16803 was used in this study. Sequences used for the alignments include DENV1 WestPac-74 (U88535.1), DENV2 S-16803 (GU289914.1), DENV3 3001 (JQ411814.1), DENV4 Sri Lanka-92 (KJ160504.1), YFV 17D (NC_002031.1), SLEV Kern217 (NC_007580.2), JEV (NC_001437.1), USUV Vienna-2001 (NC_006551.1), MVEV (NC_000943.1), WNV-1 NY99 (NC_009942.1), and ZIKV MR-766 (NC_012532.1).

DENV Reverse Genetics: DENV2 S16803 was used in this study. Recombinant viruses were created using a four-plasmid system as previously described (Messer et al. 2012 PLoS Negl Trop Dis 6(2):e1486), wherein the DENV genome was split into four segments, each cloned into a separate plasmid. The DENV plasmids were digested and ligated to form a single template for in vitro transcription. The resulting RNA was electroporated into either C6/36 or Vero cells. Virus-containing supernatant was harvested at 4-5 days post electroporation and passaged. DENV variants were created through site-directed mutagenesis of the DENV plasmids.

Library Generation and Directed Evolution: DENV fusion loop libraries were generated through saturation mutagenesis of the indicated resides, based on a previously published protocol (Tse et al. 2017 PNAS 114:E4812-E4821). Degenerate NNK oligonucleotides were used to amplify the region, generating a library of mutated DNA fragments. Q5 DNA Polymerase was used with less than 18 cycles to maintain accuracy. The resulting library was cloned into the DENV reverse genetics system. The ligated plasmids were electroporated into DH10B ElectroMax cells (Invitrogen) and directly plated on 5,245 mm² dishes (Corning) to avoid bias from suspension culture. Colonies were pooled and purified using a Maxiprep kit (Qiagen), and the plasmid library used for DENV reverse genetics (above). Viral libraries were passaged three times in the corresponding cell type.

High-throughput Sequencing and Analysis: Viral RNA was isolated with a QIAamp viral RNA kit (Qiagen), and cDNA produced using the Superscript IV Reverse Transcriptase (Invitrogen). Amplicons were prepared for sequencing using the Illumina TruSeq system with two rounds of PCR using Q5 Hot Start DNA polymerase (NEB). For the first round of PCR, primers were specific to the DENV2 E sequence surrounding the fusion loop motif with overhangs for the Illumina adapters. After purification, this product was used as the template for the second round of PCR using Illumina P5 and P7 primers containing 8-nucleotide indexes. Purified PCR products were analyzed on a Bioanalyzer (Agilent Technologies) and quantified on a Qubit 4 fluorometer (Invitrogen). Amplicon libraries were run on a MiSeq system with 2×150 bp reads. Plasmid and P0 libraries were sequenced at a depth of ˜4.5 million reads; later passages were sequenced at a depth of ˜750,000 reads. Custom perl and R scripts were used to analyze and plot the data as previously published (Tse et al. 2022 mBio 13:1-18).

DENV Growth Kinetics: One day before infection, 5×10⁵ cells were seeded in every well of a 6-well plate. Cells with infected with an MOI of 0.05-0.1, assuming 1×10⁶ cells on the day of infection. Infection was carried out for one hour in the incubator, followed by 3× washes with PBS and replenishment with fresh infection medium. 300 uL of viral supernatant was collected at 0, 24, 48, 72, 96, and 120 hours and stored at −80° C. All experiments were performed independently at least three times.

DENV Focus-Forming Assay: Titers of viral supernatant were determined using a standard DENV focus-forming assay. In brief, cells were seeded at 2×10⁴ cells per well of a 96-well plate one day before infection. The next day, 50 uL of 10-fold serial dilution of viral supernatant were added to each well for 1 hour in the incubator. After, 125 uL of overlay (Opti-MEM, 2% FBS, NEAA, P/S, and methylcellulose) was added to each well. Infection was allowed to continue for 48 hours in the incubator. Overlay was removed, and each well rinsed 3× with PBS followed by a 30 minute fixation with 10% formalin in PBS. Cells were blocked in permeabilization buffer (eBioscience) with 5% nonfat dried milk. Primary Abs anti-prM 2H2 and anti-E 4G2 from nonpurified hybridoma supernatant were used at a 1:500 dilution in blocking buffer. Goat anti-mouse HRP secondary (SeraCare KPL) were used at a 1:1000 dilution in blocking buffer. Followed washing, foci were developed using TrueBlue HRP substrate (SeraCare) and counted using an automated Immunospot analyzer (Cellular Technology).

Thermal Stability Assay: The indicated viruses were thawed and incubated at temperatures ranging from 4° C. to 55° C. for one hour. Following, viral titers were determined by focus-forming assay as described above.

Western Blotting: Viral supernatants were combined with 4× Laemmli Sample Buffer (Bio-Rad) and boiled at 95° C. for 5 minutes. After SDS-PAGE electrophoresis, samples were transferred to PVDF membrane and blocked in 3% nonfat milk in PBS-T. A polyclonal rabbit anti-prM (1:1000; Invitrogen PA5-34966) and polyclonal rabbit anti-Env (Invitrogen PA5-32246) in 2% BSA in PBS-T were incubated on the blot for 1 hour at 37C. A combination of goat anti-rabbit HRP (1:10,000 Jackson ImmunoLab) and sheep anti-human HRP (1:5000, GE Healthcare) in 3% milk in PBS-T were incubated on the blot for 1 hour at room temperature. Blots were developed by SuperSignal West Pico Plus chemiluminescent substrate (ThermoFisher). Blots were imaged on an iBright FL1500 imaging system (Invitrogen).

FRNT Assay: Focus reduction neutralization titer (FRNT) assays were performed as described previously with C6/36 cells. 1×10⁵ cells were seeded in a 96-well plate the day prior to infection. Abs or sera were serially diluted and mixed with virus (˜100 FFU/well) at a 1:1 volume and incubated for 1 hour in the incubator. The mixture was added onto the plate with cells and incubated for 1 hour in the incubator, then overlay was added (see Focus-Forming Assay) and plates were incubated for 48 hours. Viral foci were stained and counted as described above (Focus-Forming Assay). A variable slope sigmoidal dose-response curve was fitted to the data, and FRNT50 values calculated with top or bottom restraints of 100 and 0 using GraphPad Prism version 9.0. All experiments were performed independently at least two times, due to limited amounts of human serum.

Statistical Analysis: GraphPad Prism version 9.0 was used for statistical analysis. Variants were compared to the wild type using Student's t test. Significant symbols are as follows: *, P<0.05; **, P<0.005; ***, P<0.0005; ****, P<0.00005. The data are graphed as means standard deviations.

### Sequences

[wt DENV2 E]
SEQ ID NO: 1
MRCIGISNRDFVEGVSGGSWVDIVLEHGSCVTTMAKNKPTLDFELIKTEAKQPATLR
KYCIEAKLTNTTTESRCPTQGEPSLNEEQDKRFVCKHSMVDRGWGNGCGLFGKGGI
VTCAMFTCKKNMEGKVVQPENLEYTIVVTPHSGEEHAVGNDTGKHGKEIKVTPQSS
ITEAELTGYGTVTMECSPRTGLDFNEMVLLQMENKAWLVHRQWFLDLPLPWLPGA
DTQGSNWIQKETLVTFKNPHAKKQDVVVLGSQEGAMHTALTGATEIQMSSGNLLFT
GHLKCRLRMDKLQLKGMSYSMCTGKFKVVKEIAETQHGTIVIRVQYEGDGSPCKIPF
EIMDLEKRHVLGRLITVNPIVTEKDSPVNIEAEPPFGDSYIIIGVDPGQLKLNWFKKGS
SIGQMFETTMRGAKRMAILGDTAWDFGSLGGVFTSIGKALHQVFGAIYGAAFSGVS
WTMKILIGVIITWIGMNSRSTSLSVSLVLVGIVTLYLGVMVQA.
[wt DENV2 E]
SEQ ID NO: 2
ATGCGTTGCATAGGAATATCAAATAGAGACTTTGTAGAAGGGGTTTCAGGAGGA
AGCTGGGTTGACATAGTCTTAGAACATGGAAGCTGCGTGACGACGATGGCAAAA
AACAAACCAACATTGGATTTTGAACTGATAAAAACAGAAGCCAAACAGCCTGCC
ACCCTAAGGAAGTACTGTATAGAGGCAAAGCTAACCAACACAACAACAGAATCT
CGCTGCCCAACACAAGGGGAACCCAGCCTAAATGAAGAGCAGGACAAAAGGTT
CGTCTGCAAACACTCCATGGTAGACAGAGGATGGGGAAATGGATGTGGATTGTT
TGGGAAGGGAGGCATTGTGACCTGTGCTATGTTTACATGCAAAAAGAACATGGA
AGGAAAAGTTGTGCAACCAGAAAACTTGGAATACACCATTGTGGTAACACCTCA
CTCAGGGGAAGAGCATGCGGTCGGAAATGACACAGGAAAACATGGCAAGGAAA
TCAAAGTAACACCGCAGAGTTCCATCACAGAAGCAGAATTGACAGGCTATGGCA
CTGTCACGATGGAGTGCTCTCCGAGAACAGGCCTCGACTTCAATGAGATGGTGTT
GCTGCAGATGGAAAACAAAGCTTGGCTGGTGCACAGGCAATGGTTCCTAGACCT
GCCGTTACCATGGCTGCCCGGAGCGGATACACAAGGGTCAAATTGGATACAGAA
AGAAACATTGGTCACTTTCAAAAATCCCCATGCGAAGAAACAGGATGTTGTTGTT
TTAGGATCCCAAGAAGGGGCCATGCACACAGCACTCACAGGGGCCACAGAAATC
CAAATGTCATCAGGAAATTTACTCTTCACAGGACATCTCAAGTGCAGGCTGAGA
ATGGACAAGCTACAGCTCAAAGGAATGTCATACTCTATGTGCACAGGAAAGTTT
AAAGTTGTGAAGGAAATAGCAGAAACACAACATGGAACAATAGTTATCAGAGTG
CAATATGAAGGGGACGGCTCTCCATGTAAAATCCCTTTTGAGATAATGGATTTGG
AAAAAAGACATGTCTTAGGTCGCCTGATCACAGTCAACCCAATTGTGACAGAAA
AAGATAGCCCAGTCAACATAGAAGCAGAACCTCCATTCGGAGACAGCTACATCA
TCATAGGAGTAGACCCGGGACAACTGAAGCTCAACTGGTTTAAGAAAGGAAGTT
CTATCGGCCAAATGTTTGAGACAACAATGAGGGGGGCGAAGAGAATGGCCATTT
TGGGTGACACAGCCTGGGATTTTGGATCCCTGGGAGGAGTGTTTACATCTATAGG
AAAAGCTCTCCACCAAGTCTTTGGAGCAATCTATGGAGCCGCCTTCAGTGGGGTT
TCATGGACTATGAAAATCCTCATAGGAGTCATTATCACATGGATAGGAATGAATT
CACGCAGCACCTCACTGTCTGTGTCACTAGTATTGGTGGGAATTGTGACACTGTA
TTTGGGAGTCATGGTGCAGGCC.
[wt DENV1 E] GenBank ® U88535.1
SEQ ID NO: 3
MRCVGIGNRDFVEGLSGATWVDVVLEHGSCVTTMAKDKPTLDIELLKTEVTNPAVL
RKLCIEAKISNTTTDSRCPTQGEATLVEEQDTNFVCRRTFVDRGWGNGCGLFGKGSL
ITCAKFKCVTKLEGKIVQYENLKYSVIVTVHTGDQHQVGNETTEHGTTATITPQAPTS
EIQLTDYGALTLDCSPRTGLDFNEMVLLTMEKKSWLVHKQWFLDLPLPWTSGASTS
QETWNRQDLLVTFKTAHAKKQEVVVLGSQEGAMHTALTGATEIQTSGTTTIFAGHL
KCRLKMDKLTLKGMSYVMCTGSFKLEKEVAETQHGTVLVQVKYEGTDAPCKIPFSS
QDEKGVTQNGRLITANPIVTDKEKPVNIEAEPPFGESYIVVGAGEKALKLSWFKKGSS
IGKMFEATARGARRMAILGDTAWDFGSIGGVFTSVGKLIHQIFGTAYGVLFSGVSWT
MKIGIGILLTWLGLNSRSTSLSMTCIAVGMVTLYLGVMVQADSGCVINWKGRELKC
GSGIFVTNEVHTWTEQYKFQADSPKRLSAAIGKAWEEGVCGIRSATRLENIMWKQIS
NELNHILLENDMKFTVVVGDVSGILAQGKKMIRPQPMEHKYSWKSWGKAKIIGADV
QNTTFIIDGPNTPECPDNQRAWNIWEVE
[wt DENV3 E] GenBank ® JQ411814.1
SEQ ID NO: 4
MRCVGIGNRDFVEGLSGATWVDVVLEHGGCVTTMAKNKPTLDIELQKTEATQLATL
RKLCIEGKITNITTDSRCPTQGEAVLPEEQDQNYVCKHTYVDRGWGNGCGLFGKGSL
VTCAKFQCLEPIEGKVVQYENLKYTVIITVHTGDQHQVGNETQGVTAEITPQASTTE
AILPEYGTLGLECSPRTGLDFNEMILLTMKNKAWMVHRQWFFDLPLPWTSGATTET
PTWNRKELLVTFKNAHAKKQEVVVLGSQEGAMHTALTGATEIQNSGGTSIFAGHLK
CRLKMDKLELKGMSYAMCTNTFVLKKEVSETQHGTILIKVEYKGEDAPCKIPFSTED
GQGKAHNGRLITANPVVTKKEEPVNIEAEPPFGESNIVIGIGDNALKINWYKKGSSIG
KMFEATARGARRMAILGDTAWDFGSVGGVLNSLGKMVHQIFGSAYTALFSGVSWV
MKIGIGVLLTWIGLNSKNTSMSFSCIAIGIITLYLGAVVQADMGCVINWKGKELKCGS
GIFVTNEVHTWTEQYKFQADSPKRLATAIAGAWENGVCGIRSTTRMENLLWKQIAN
ELNYILWENNIKLTVVVGDTIGVLEQGKRTLTPQPMELKYSWKTWGKAKIVTAETQ
NSSFIIDGPNTPECPSASRAWNVWEVE
[wt DENV4 E] GenBank ® KJ160504.1
SEQ ID NO: 5
MRCVGVGNRDFVEGVSGGAWVDLVLEHGGCVTTMAQGKPTLDFELTKTTAKEVA
LLRTYCIEASISNITTATRCPTQGEPYLKEEQDQQYICRRDVVDRGWGNGCGLFGKG
GVVTCAKFSCSGKITGNLVQIENLEYTVVVTVHNGDTHAVGNDTSNHGVTATITPRS
PSVEVKLPDYGELTLDCEPRSGIDFNEMILMKMKKKTWLVHKQWFLDLPLPWTAGA
DTSEVHWNYKERMVTFKVPHAKRQDVTVLGSQEGAMHSALAGATEVDSGDGNHM
FAGHLKCKVRMEKLRIKGMSYTMCSGKFSIDKEMAETQHGTTVVKVKYEGAGAPC
KVPIEIRDVNKEKVVGRVISSTPLAENTNSVTNIELEPPFGDSYIVIGVGNSALTLHWF
RKGSSIGKMFESTYRGAKRMAILGETAWDFGSVGGLFTSLGKAVHQVFGSVYTTMF
GGVSWMIRILIGFLVLWIGTNSRNTSMAMTCIAVGGITLFLGFTVQA
[substituted DENV2 E]
SEQ ID NO: 6
MRCIGISNRDFVEGVSGGSWVDIVLEHGSCVTTMAKNKPTLDFELIKTEAKQPATLR
KYCIEAKLTNTTTESRCPTQGEPSLNEEQDKRFVCKHSMVDRGWGSGCLLFGKGGIV
TCAMFTCKKNMEGKVVQPENLEYTIVVTPHSGEEHAVGNDTGKHGKEIKVTPQSSIT
EAELTGYGTVTMECSPRTGLDFNEMVLLQMENKAWLVHRQWFLDLPLPWLPGADT
QGSNWIQKETLVTFKNPHAKKQDVVVLGSQEGAMHTALTGATEIQMSSGNLLFTGH
LKCRLRMDKLQLKGMSYSMCTGKFKVVKEIAETQHGTIVIRVQYEGDGSPCKIPFEI
MDLEKRHVLGRLITVNPIVTEKDSPVNIEAEPPFGDSYIIIGVDPGQLKLNWFKKGSSI
GQMFETTMRGAKRMAILGDTAWDFGSLGGVFTSIGKALHQVFGAIYGAAFSGVSW
TMKILIGVIITWIGMNSRSTSLSVSLVLVGIVTLYLGVMVQA.
[substituted DENV1 E]
SEQ ID NO: 7
MRCVGIGNRDFVEGLSGATWVDVVLEHGSCVTTMAKDKPTLDIELLKTEVTNPAVL
RKLCIEAKISNTTTDSRCPTQGEATLVEEQDTNFVCRRTFVDRGWGSGCLLFGKGSLI
TCAKFKCVTKLEGKIVQYENLKYSVIVTVHTGDQHQVGNETTEHGTTATITPQAPTS
EIQLTDYGALTLDCSPRTGLDFNEMVLLTMEKKSWLVHKQWFLDLPLPWTSGASTS
QETWNRQDLLVTFKTAHAKKQEVVVLGSQEGAMHTALTGATEIQTSGTTTIFAGHL
KCRLKMDKLTLKGMSYVMCTGSFKLEKEVAETQHGTVLVQVKYEGTDAPCKIPFSS
QDEKGVTQNGRLITANPIVTDKEKPVNIEAEPPFGESYIVVGAGEKALKLSWFKKGSS
IGKMFEATARGARRMAILGDTAWDFGSIGGVFTSVGKLIHQIFGTAYGVLFSGVSWT
MKIGIGILLTWLGLNSRSTSLSMTCIAVGMVTLYLGVMVQADSGCVINWKGRELKC
GSGIFVTNEVHTWTEQYKFQADSPKRLSAAIGKAWEEGVCGIRSATRLENIMWKQIS
NELNHILLENDMKFTVVVGDVSGILAQGKKMIRPQPMEHKYSWKSWGKAKIIGADV
QNTTFIIDGPNTPECPDNQRAWNIWEVE
[substituted DENV3 E]
SEQ ID NO: 8
MRCVGIGNRDFVEGLSGATWVDVVLEHGGCVTTMAKNKPTLDIELQKTEATQLATL
RKLCIEGKITNITTDSRCPTQGEAVLPEEQDQNYVCKHTYVDRGWGSGCLLFGKGSL
VTCAKFQCLEPIEGKVVQYENLKYTVIITVHTGDQHQVGNETQGVTAEITPQASTTE
AILPEYGTLGLECSPRTGLDFNEMILLTMKNKAWMVHRQWFFDLPLPWTSGATTET
PTWNRKELLVTFKNAHAKKQEVVVLGSQEGAMHTALTGATEIQNSGGTSIFAGHLK
CRLKMDKLELKGMSYAMCTNTFVLKKEVSETQHGTILIKVEYKGEDAPCKIPFSTED
GQGKAHNGRLITANPVVTKKEEPVNIEAEPPFGESNIVIGIGDNALKINWYKKGSSIG
KMFEATARGARRMAILGDTAWDFGSVGGVLNSLGKMVHQIFGSAYTALFSGVSWV
MKIGIGVLLTWIGLNSKNTSMSFSCIAIGIITLYLGA VVQADMGCVINWKGKELKCGS
GIFVTNEVHTWTEQYKFQADSPKRLATAIAGAWENGVCGIRSTTRMENLLWKQIAN
ELNYILWENNIKLTVVVGDTIGVLEQGKRTLTPQPMELKYSWKTWGKAKIVTAETQ
NSSFIIDGPNTPECPSASRAWNVWEVE
[substituted DENV4 E]
SEQ ID NO: 9
MRCVGVGNRDFVEGVSGGAWVDLVLEHGGCVTTMAQGKPTLDFELTKTTAKEVA
LLRTYCIEASISNITTATRCPTQGEPYLKEEQDQQYICRRDVVDRGWGSGCLLFGKGG
VVTCAKFSCSGKITGNLVQIENLEYTVVVTVHNGDTHAVGNDTSNHGVTATITPRSP
SVEVKLPDYGELTLDCEPRSGIDFNEMILMKMKKKTWLVHKQWFLDLPLPWTAGA
DTSEVHWNYKERMVTFKVPHAKRQDVTVLGSQEGAMHSALAGATEVDSGDGNHM
FAGHLKCKVRMEKLRIKGMSYTMCSGKFSIDKEMAETQHGTTVVKVKYEGAGAPC
KVPIEIRDVNKEKVVGRVISSTPLAENTNSVTNIELEPPFGDSYIVIGVGNSALTLHWF
RKGSSIGKMFESTYRGAKRMAILGETAWDFGSVGGLFTSLGKAVHQVFGSVYTTMF
GGVSWMIRILIGFLVLWIGTNSRNTSMAMTCIAVGGITLFLGFTVQA
[modified E fusion loop epitopel
SEQ ID NO: 10
DRGWGSGCLLFGK.
[modified prM cleavage site]
SEQ ID NO: 11
TGAGRRSKRS.
[DV2-prM] GenBank: GU289914.1
SEQ ID NO: 12
FHLTTRNGEPHMIVSRQEKGKSLLFKTEDGVNMCTLMAMDLGELCEDTITYNCPLL
RQNEPEDIDCWCNSTSTWVTYGTCTTTGEHRREKRSVALVPHVGMGLETRTETWM
SSEGAWKHAQRIETWILRHPGFTIMAAILAYTIGTTHFQRALIFILLTAVAPSMT.
Library 1 consensus
SEQ ID NO: 13
DRGXGXGXXXFGK.
Library 2 consensus
SEQ ID NO: 14
DRGXXXXXGLFGK.
VT151 DNA
SEQ ID NO: 15
ATGCGTTGCATAGGAATATCAAATAGAGACTTTGTAGAAGGGGTTTCAGGAGGA
AGCTGGGTTGACATAGTCTTAGAACATGGAAGCTGCGTGACGACGATGGCAAAA
AACAAACCAACATTGGATTTTGAACTGATAAAAACAGAAGCCAAACAGCCTGCC
ACCCTAAGGAAGTACTGTATAGAGGCAAAGCTAACCAACACAACAACAGAATCT
CGCTGCCCAACACAAGGGGAACCCAGCCTAAATGAAGAGCAGGACAAAAGGTT
CGTCTGCAAACACTCCATGGTAGACCGCGGATGGGGATCTGGATGTTTGCTGTTT
GGGAAGGGAGGCATTGTGACCTGTGCTATGTTTACATGCAAAAAGAACATGGAA
GGAAAAGTTGTGCAACCAGAAAACTTGGAATACACCATTGTGGTAACACCTCAC
TCAGGGGAAGAGCATGCGGTCGGAAATGACACAGGAAAACATGGCAAGGAAAT
CAAAGTAACACCGCAGAGTTCCATCGCAGAAGCAGAATTGACAGGCTATGGCAC
GGTCACCATGGAGTGCTCTCCGAGAACAGGCCTCGACTTCAATGAGATGGTGTTG
CTGCAGATGGAAAACAAAGCTTGGCTGGTGCACAGGCAATGGTTCCTAGACCTG
CCGTTACCATGGCTGCCCGGAGCGGATACACAAGGGTCAAATTGGATACAGAAA
GAAACATTGGTCACTTTCAAAAATCCCCATGCGAAGAAACAGGATGTTGTTGTTT
TAGGATCCCAAGAAGGGGCCATGCACACAGCACTCACAGGGGCCACAGAAATCC
AAATGTCATCAGGAAATTTACTCTTCACAGGACATCTCAAGTGCAGGCTGAGAAT
GGACAAGCTACAGCTCAAAGGAATGTCATACTCTATGTGCACAGGAAAGTTTAA
AGTIGTGAAGGAAATAGCAGAAACACAACATGGAACAATAGTTATCAGAGTGCA
ATATGAAGGGGACGGCTCTCCATGTAAAATCCCTTTTGAGATAATGGATTTGGAA
AAAAGACATGTCTTAGGTCGCCTGATCACAGTCAACCCAATTGTGACAGAAAAA
GATAGCCCAGTCAACATAGAAGCAGAACCTCCATTCGGAGACAGCTACATCATC
ATAGGAGTAGACCCGGGACAACTGAAGCTCAACTGGTTTAAGAAAGGAAGTTCT
ATCGGCCAAATGTTTGAGACAACAATGAGGGGGGCGAAGAGAATGGCCATTTTG
GGTGACACAGCCTGGGATTTTGGATCCCTGGGAGGAGTGTTTACATCTATAGGAA
AAGCTCTCCACCAAGTCTTTGGAGCAATCTATGGAGCCGCCTTCAGTGGGGTTTC
ATGGACTATGAAAATCCTCATAGGAGTCATTATCACATGGATAGGAATGAATTCA
CGCAGCACCTCACTGTCTGTGTCACTAGTATTGGTGGGAATTGTGACACTGTATT
TGGGAGTCATGGTGCAGGCC.
VT151 amino acid
SEQ ID NO: 16
MRCIGISNRDFVEGVSGGSWVDIVLEHGSCVTTMAKNKPTLDFELIKTEAKQPATLR
KYCIEAKLTNTTTESRCPTQGEPSLNEEQDKRFVCKHSMVDRGWGSGCLLFGKGGIV
TCAMFTCKKNMEGKVVQPENLEYTIVVTPHSGEEHAVGNDTGKHGKEIKVTPQSSI
AEAELTGYGTVTMECSPRTGLDFNEMVLLQMENKAWLVHRQWFLDLPLPWLPGAD
TQGSNWIQKETLVTFKNPHAKKQDVVVLGSQEGAMHTALTGATEIQMSSGNLLFTG
HLKCRLRMDKLQLKGMSYSMCTGKFKVVKEIAETQHGTIVIRVQYEGDGSPCKIPFE
IMDLEKRHVLGRLITVNPIVTEKDSPVNIEAEPPFGDSYIIIGVDPGQLKLNWFKKGSSI
GQMFETTMRGAKRMAILGDTAWDFGSLGGVFTSIGKALHQVFGAIYGAAFSGVSW
TMKILIGVIITWIGMNSRSTSLSVSLVLVGIVTLYLGVMVQA.
VT163 amino acid = VT151 + prM mutations
SEQ ID NO: 17
FHLTTRNGEPHMIVSRQEKGKSLLFKTEDGVNMCTLMAMDLGELCEDTITYNCPLL
RQNEPEDIDCWCNSTSTWVTYGTCTTTGAGRRSKRSVALVPHVGMGLETRTETWM
SSEGAWKHAQRIETWILRHPGFTIMAAILAYTIGTTHFQRALIFILLTAVAPSMT.

### Tables

TABLE 1

| | Abbreviation | |
|---|---|---|
| Amino Acid Residue | Three-Letter Code | One-Letter Code |
| Alanine | Ala | A |
| Arginine | Arg | R |
| Asparagine | Asn | N |
| Aspartic acid (Aspartate) | Asp | D |
| Cysteine | Cys | C |
| Glutamine | Gln | Q |
| Glutamic acid (Glutamate) | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

TABLE 2

| | |
|---|---|
| Modified Amino Acid Residue | Abbreviation |

Amino Acid Residue Derivatives

| | |
|---|---|
| 2-Aminoadipic acid | Aad |
| 3-Aminoadipic acid | bAad |
| beta-Alanine, beta-Aminoproprionic acid | bAla |
| 2-Aminobutyric acid | Abu |
| 4-Aminobutyric acid, Piperidinic acid | 4Abu |
| 6-Aminocaproic acid | Acp |
| 2-Aminoheptanoic acid | Ahe |
| 2-Aminoisobutyric acid | Aib |
| 3-Aminoisobutyric acid | bAib |
| 2-Aminopimelic acid | Apm |
| t-butylalanine | t-BuA |
| Citrulline | Cit |
| Cyclohexylalanine | Cha |
| 2,4-Diaminobutyric acid | Dbu |
| Desmosine | Des |
| 2,2′-Diaminopimelic acid | Dpm |
| 2,3-Diaminoproprionic acid | Dpr |
| N-Ethylglycine | EtGly |
| N-Ethylasparagine | EtAsn |
| Homoarginine | hArg |
| Homocysteine | hCys |
| Homoserine | hSer |
| Hydroxylysine | Hyl |
| Allo-Hydroxylysine | aHyl |
| 3-Hydroxyproline | 3Hyp |
| 4-Hydroxyproline | 4Hyp |
| Isodesmosine | Ide |
| allo-Isoleucine | aIle |
| Methionine sulfoxide | MSO |
| N-Methylglycine, sarcosine | MeGly |
| N-Methylisoleucine | MeIle |
| 6-N-Methyllysine | MeLys |
| N-Methylvaline | MeVal |
| 2-Naphthylalanine | 2-Nal |
| Norvaline | Nva |
| Norleucine | Nle |
| Ornithine | Orn |
| 4-Chlorophenylalanine | Phe(4-Cl) |
| 2-Fluorophenylalanine | Phe(2-F) |
| 3-Fluorophenylalanine | Phe(3-F) |
| 4-Fluorophenylalanine | Phe(4-F) |
| Phenylglycine | Phg |
| Beta-2-thienylalanine | Thi |

1. A recombinant flavivirus E glycoprotein comprising a flavivirus E glycoprotein backbone comprising a fusion loop epitope, and the following amino acid substitutions in the fusion loop epitope, wherein the numbering corresponds to the amino acid sequence of SEQ ID NO: 1,
103S and 106L.

2-4. (canceled)

5. The recombinant flavivirus E glycoprotein of claim 1, further comprising one or more amino acid substitution(s) in position 101, 102, 104, 105, and/or 107.

6. The recombinant flavivirus E glycoprotein of claim 5, wherein the one or more amino acid substitution(s) comprise 101W, 102G, 104G, 105C, and/or 107L, in any combination.

7. (canceled)

8. The recombinant flavivirus E glycoprotein of claim 1, wherein the E glycoprotein backbone is a backbone of dengue virus 1, 2, 3, 4, or any derivative thereof.

9. The recombinant flavivirus E glycoprotein of claim 1, wherein the fusion loop epitope comprises the following amino acid sequence in positions 98-110, wherein the numbering corresponds to the amino acid sequence of SEQ ID NO: 1:
DRGWGSGCLLFGK (SEQ ID NO:10).

10. The recombinant flavivirus E glycoprotein of claim 1, wherein the flavivirus E glycoprotein backbone comprises the amino acid sequence of SEQ ID NO:3, SEQ ID NO:1, SEQ ID NO:4, SEQ ID NO:5 or a sequence at least about 90% identical thereto.

11. The recombinant flavivirus E glycoprotein of claim 10, comprising the amino acid sequence of SEQ ID NO:7, SEQ ID NO:6, SEQ ID NO: 8, or SEQ ID NO:9.

12-17. (canceled)

18. An isolated nucleic acid molecule encoding the E glycoprotein of claim 1.

19. The isolated nucleic acid molecule of claim 18, further encoding a modified flavivirus prM glycoprotein, wherein the modified prM glycoprotein comprises a furin cleavage site with enhanced cleavability by a furin enzyme.

20. (canceled)

21. The isolated nucleic acid molecule of claim 19, wherein the furin cleavage site comprises one or more substitution at amino acid position 85, 86, 87, and/or 89, wherein the furin cleavage site comprises the amino acid sequence of SEQ ID NO: 11 in positions 83-92 of the wildtype (unmodified) prM glycoprotein, wherein the numbering corresponds to the amino acid sequence of SEQ ID NO:12.

22. (canceled)

23. A flavivirus particle or virus like particle (VLP) comprising the E glycoprotein of claim 1.

24. The flavivirus particle or VLP of claim 23, wherein the particle is in a mature form.

25. (canceled)

26. The flavivirus particle or VLP of claim 23, further comprising a chimeric prM glycoprotein, wherein the modified prM glycoprotein comprises a furin cleavage site with enhanced cleavability by a furin enzyme.

27. The flavivirus particle or VLP of claim 26, wherein the furin cleavage site comprises one or more substitution at amino acid position 85, 86, 87, and/or 89, wherein the furin cleavage site comprises the amino acid sequence of SEQ ID NO:11 in positions 83-92 of the wildtype prM glycoprotein, wherein the numbering corresponds to the amino acid sequence of SEQ ID NO:12.

28. (canceled)

29. An isolated nucleic acid molecule encoding the flavivirus particle or VLP of claim 23.

30. A vector comprising the isolated nucleic acid molecule of claim 18.

31-33. (canceled)

34. A composition comprising the E glycoprotein of claim 1, in a pharmaceutically acceptable carrier.

35. A method of producing an immune response to a flavivirus in a subject, comprising administering to the subject an effective amount of the E glycoprotein of claim 1.

36. A method of treating a flavivirus infection in a subject, comprising administering to the subject an effective amount of the E glycoprotein of claim 1.

37-55. (canceled)

56. A method of producing a flavivirus particle, comprising:
constructing a flavivirus particle comprising the recombinant E glycoprotein of claim 1;
thereby producing a flavivirus particle comprising the recombinant E glycoprotein.

57-58. (canceled)


SEQUENCE LISTING (19 sequences)

SEQ ID NO: 1 - protein, 495 aa, Dengue virus
  source 1..495: note = DENV2
MRCIGISNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGNGCGLFGKGGIVTCAMFTCKKNMEGKVVQPENLEYTIVVTPHSGEEHAVGNDTGKHGKEIKVTPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVDPGQLKLNWFKKGSSIGQMFETTMRGAKRMAILGDTAWDFGSLGGVFTSIGKALHQVFGAIYGAAFSGVSWTMKILIGVIITWIGMNSRSTSLSVSLVLVGIVTLYLGVMVQA

SEQ ID NO: 2 - DNA, 1485 bp, Dengue virus
  source 1..1485: mol_type = genomic DNA; serotype = DENV2
atgcgttgcataggaatatcaaatagagactttgtagaaggggtttcaggaggaagctgggttgacatagtcttagaacatggaagctgcgtgacgacgatggcaaaaaacaaaccaacattggattttgaactgataaaaacagaagccaaacagcctgccaccctaaggaagtactgtatagaggcaaagctaaccaacacaacaacagaatctcgctgcccaacacaaggggaacccagcctaaatgaagagcaggacaaaaggttcgtctgcaaacactccatggtagacagaggatggggaaatggatgtggattgtttgggaagggaggcattgtgacctgtgctatgtttacatgcaaaaagaacatggaaggaaaagttgtgcaaccagaaaacttggaatacaccattgtggtaacacctcactcaggggaagagcatgcggtcggaaatgacacaggaaaacatggcaaggaaatcaaagtaacaccgcagagttccatcacagaagcagaattgacaggctatggcactgtcacgatggagtgctctccgagaacaggcctcgacttcaatgagatggtgttgctgcagatggaaaacaaagcttggctggtgcacaggcaatggttcctagacctgccgttaccatggctgcccggagcggatacacaagggtcaaattggatacagaaagaaacattggtcactttcaaaaatccccatgcgaagaaacaggatgttgttgttttaggatcccaagaaggggccatgcacacagcactcacaggggccacagaaatccaaatgtcatcaggaaatttactcttcacaggacatctcaagtgcaggctgagaatggacaagctacagctcaaaggaatgtcatactctatgtgcacaggaaagtttaaagttgtgaaggaaatagcagaaacacaacatggaacaatagttatcagagtgcaatatgaaggggacggctctccatgtaaaatcccttttgagataatggatttggaaaaaagacatgtcttaggtcgcctgatcacagtcaacccaattgtgacagaaaaagatagcccagtcaacatagaagcagaacctccattcggagacagctacatcatcataggagtagacccgggacaactgaagctcaactggtttaagaaaggaagttctatcggccaaatgtttgagacaacaatgaggggggcgaagagaatggccattttgggtgacacagcctgggattttggatccctgggaggagtgtttacatctataggaaaagctctccaccaagtctttggagcaatctatggagccgccttcagtggggtttcatggactatgaaaatcctcataggagtcattatcacatggataggaatgaattcacgcagcacctcactgtctgtgtcactagtattggtgggaattgtgacactgtatttgggagtcatggtgcaggcc

SEQ ID NO: 3 - protein, 651 aa, Dengue virus
  source 1..651: note = DENV1
MRCVGIGNRDFVEGLSGATWVDVVLEHGSCVTTMAKDKPTLDIELLKTEVTNPAVLRKLCIEAKISNTTTDSRCPTQGEATLVEEQDTNFVCRRTFVDRGWGNGCGLFGKGSLITCAKFKCVTKLEGKIVQYENLKYSVIVTVHTGDQHQVGNETTEHGTTATITPQAPTSEIQLTDYGALTLDCSPRTGLDFNEMVLLTMEKKSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSSQDEKGVTQNGRLITANPIVTDKEKPVNIEAEPPFGESYIVVGAGEKALKLSWFKKGSSIGKMFEATARGARRMAILGDTAWDFGSIGGVFTSVGKLIHQIFGTAYGVLFSGVSWTMKIGIGILLTWLGLNSRSTSLSMTCIAVGMVTLYLGVMVQADSGCVINWKGRELKCGSGIFVTNEVHTWTEQYKFQADSPKRLSAAIGKAWEEGVCGIRSATRLENIMWKQISNELNHILLENDMKFTVVVGDVSGILAQGKKMIRPQPMEHKYSWKSWGKAKIIGADVQNTTFIIDGPNTPECPDNQRAWNIWEVE

SEQ ID NO: 4 - protein, 649 aa, Dengue virus
  source 1..649: note = DENV3
MRCVGIGNRDFVEGLSGATWVDVVLEHGGCVTTMAKNKPTLDIELQKTEATQLATLRKLCIEGKITNITTDSRCPTQGEAVLPEEQDQNYVCKHTYVDRGWGNGCGLFGKGSLVTCAKFQCLEPIEGKVVQYENLKYTVIITVHTGDQHQVGNETQGVTAEITPQASTTEAILPEYGTLGLECSPRTGLDFNEMILLTMKNKAWMVHRQWFFDLPLPWTSGATTETPTWNRKELLVTFKNAHAKKQEVVVLGSQEGAMHTALTGATEIQNSGGTSIFAGHLKCRLKMDKLELKGMSYAMCTNTFVLKKEVSETQHGTILIKVEYKGEDAPCKIPFSTEDGQGKAHNGRLITANPVVTKKEEPVNIEAEPPFGESNIVIGIGDNALKINWYKKGSSIGKMFEATARGARRMAILGDTAWDFGSVGGVLNSLGKMVHQIFGSAYTALFSGVSWVMKIGIGVLLTWIGLNSKNTSMSFSCIAIGIITLYLGAVVQADMGCVINWKGKELKCGSGIFVTNEVHTWTEQYKFQADSPKRLATAIAGAWENGVCGIRSTTRMENLLWKQIANELNYILWENNIKLTVVVGDTIGVLEQGKRTLTPQPMELKYSWKTWGKAKIVTAETQNSSFIIDGPNTPECPSASRAWNVWEVE

SEQ ID NO: 5 - protein, 495 aa, Dengue virus
  source 1..495: note = DENV4
MRCVGVGNRDFVEGVSGGAWVDLVLEHGGCVTTMAQGKPTLDFELTKTTAKEVALLRTYCIEASISNITTATRCPTQGEPYLKEEQDQQYICRRDVVDRGWGNGCGLFGKGGVVTCAKFSCSGKITGNLVQIENLEYTVVVTVHNGDTHAVGNDTSNHGVTATITPRSPSVEVKLPDYGELTLDCEPRSGIDFNEMILMKMKKKTWLVHKQWFLDLPLPWTAGADTSEVHWNYKERMVTFKVPHAKRQDVTVLGSQEGAMHSALAGATEVDSGDGNHMFAGHLKCKVRMEKLRIKGMSYTMCSGKFSIDKEMAETQHGTTVVKVKYEGAGAPCKVPIEIRDVNKEKVVGRVISSTPLAENTNSVTNIELEPPFGDSYIVIGVGNSALTLHWFRKGSSIGKMFESTYRGAKRMAILGETAWDFGSVGGLFTSLGKAVHQVFGSVYTTMFGGVSWMIRILIGFLVLWIGTNSRNTSMAMTCIAVGGITLFLGFTVQA

SEQ ID NO: 6 - protein, 495 aa, synthetic construct
  source 1..495: note = substituted DENV2 E glycoprotein
MRCIGISNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGSGCLLFGKGGIVTCAMFTCKKNMEGKVVQPENLEYTIVVTPHSGEEHAVGNDTGKHGKEIKVTPQSSITEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVDPGQLKLNWFKKGSSIGQMFETTMRGAKRMAILGDTAWDFGSLGGVFTSIGKALHQVFGAIYGAAFSGVSWTMKILIGVIITWIGMNSRSTSLSVSLVLVGIVTLYLGVMVQA

SEQ ID NO: 7 - protein, 651 aa, Synthetic construct
  source 1..651: note = substituted DENV1 E glycoprotein
MRCVGIGNRDFVEGLSGATWVDVVLEHGSCVTTMAKDKPTLDIELLKTEVTNPAVLRKLCIEAKISNTTTDSRCPTQGEATLVEEQDTNFVCRRTFVDRGWGSGCLLFGKGSLITCAKFKCVTKLEGKIVQYENLKYSVIVTVHTGDQHQVGNETTEHGTTATITPQAPTSEIQLTDYGALTLDCSPRTGLDFNEMVLLTMEKKSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSSQDEKGVTQNGRLITANPIVTDKEKPVNIEAEPPFGESYIVVGAGEKALKLSWFKKGSSIGKMFEATARGARRMAILGDTAWDFGSIGGVFTSVGKLIHQIFGTAYGVLFSGVSWTMKIGIGILLTWLGLNSRSTSLSMTCIAVGMVTLYLGVMVQADSGCVINWKGRELKCGSGIFVTNEVHTWTEQYKFQADSPKRLSAAIGKAWEEGVCGIRSATRLENIMWKQISNELNHILLENDMKFTVVVGDVSGILAQGKKMIRPQPMEHKYSWKSWGKAKIIGADVQNTTFIIDGPNTPECPDNQRAWNIWEVE

SEQ ID NO: 8 - protein, 649 aa, synthetic construct
  source 1..649: note = substituted DENV3 E glycoprotein
MRCVGIGNRDFVEGLSGATWVDVVLEHGGCVTTMAKNKPTLDIELQKTEATQLATLRKLCIEGKITNITTDSRCPTQGEAVLPEEQDQNYVCKHTYVDRGWGSGCLLFGKGSLVTCAKFQCLEPIEGKVVQYENLKYTVIITVHTGDQHQVGNETQGVTAEITPQASTTEAILPEYGTLGLECSPRTGLDFNEMILLTMKNKAWMVHRQWFFDLPLPWTSGATTETPTWNRKELLVTFKNAHAKKQEVVVLGSQEGAMHTALTGATEIQNSGGTSIFAGHLKCRLKMDKLELKGMSYAMCTNTFVLKKEVSETQHGTILIKVEYKGEDAPCKIPFSTEDGQGKAHNGRLITANPVVTKKEEPVNIEAEPPFGESNIVIGIGDNALKINWYKKGSSIGKMFEATARGARRMAILGDTAWDFGSVGGVLNSLGKMVHQIFGSAYTALFSGVSWVMKIGIGVLLTWIGLNSKNTSMSFSCIAIGIITLYLGAVVQADMGCVINWKGKELKCGSGIFVTNEVHTWTEQYKFQADSPKRLATAIAGAWENGVCGIRSTTRMENLLWKQIANELNYILWENNIKLTVVVGDTIGVLEQGKRTLTPQPMELKYSWKTWGKAKIVTAETQNSSFIIDGPNTPECPSASRAWNVWEVE

SEQ ID NO: 9 - protein, 495 aa, synthetic construct
  source 1..495: note = substituted DENV4 E glycoprotein
MRCVGVGNRDFVEGVSGGAWVDLVLEHGGCVTTMAQGKPTLDFELTKTTAKEVALLRTYCIEASISNITTATRCPTQGEPYLKEEQDQQYICRRDVVDRGWGSGCLLFGKGGVVTCAKFSCSGKITGNLVQIENLEYTVVVTVHNGDTHAVGNDTSNHGVTATITPRSPSVEVKLPDYGELTLDCEPRSGIDFNEMILMKMKKKTWLVHKQWFLDLPLPWTAGADTSEVHWNYKERMVTFKVPHAKRQDVTVLGSQEGAMHSALAGATEVDSGDGNHMFAGHLKCKVRMEKLRIKGMSYTMCSGKFSIDKEMAETQHGTTVVKVKYEGAGAPCKVPIEIRDVNKEKVVGRVISSTPLAENTNSVTNIELEPPFGDSYIVIGVGNSALTLHWFRKGSSIGKMFESTYRGAKRMAILGETAWDFGSVGGLFTSLGKAVHQVFGSVYTTMFGGVSWMIRILIGFLVLWIGTNSRNTSMAMTCIAVGGITLFLGFTVQA

SEQ ID NO: 10 - protein, 13 aa, synthetic construct
  source 1..13: note = modified E fusion loop epitope
DRGWGSGCLLFGK

SEQ ID NO: 11 - protein, 10 aa, synthetic construct
  source 1..10: note = modififed prM cleavage site
TGAGRRSKRS

SEQ ID NO: 12 - protein, 166 aa, Dengue virus
  source 1..166: note = DENV2
FHLTTRNGEPHMIVSRQEKGKSLLFKTEDGVNMCTLMAMDLGELCEDTITYNCPLLRQNEPEDIDCWCNSTSTWVTYGTCTTTGEHRREKRSVALVPHVGMGLETRTETWMSSEGAWKHAQRIETWILRHPGFTIMAAILAYTIGTTHFQRALIFILLTAVAPSMT

SEQ ID NO: 13 - protein, 13 aa, synthetic construct
DRGXGXGXXXFGK

SEQ ID NO: 14 - protein, 13 aa, synthetic construct
DRGXXXXXGLFGK

SEQ ID NO: 15 - DNA, 1485 bp, synthetic construct
  source 1..1485: mol_type = other DNA
atgcgttgcataggaatatcaaatagagactttgtagaaggggtttcaggaggaagctgggttgacatagtcttagaacatggaagctgcgtgacgacgatggcaaaaaacaaaccaacattggattttgaactgataaaaacagaagccaaacagcctgccaccctaaggaagtactgtatagaggcaaagctaaccaacacaacaacagaatctcgctgcccaacacaaggggaacccagcctaaatgaagagcaggacaaaaggttcgtctgcaaacactccatggtagaccgcggatggggatctggatgtttgctgtttgggaagggaggcattgtgacctgtgctatgtttacatgcaaaaagaacatggaaggaaaagttgtgcaaccagaaaacttggaatacaccattgtggtaacacctcactcaggggaagagcatgcggtcggaaatgacacaggaaaacatggcaaggaaatcaaagtaacaccgcagagttccatcgcagaagcagaattgacaggctatggcacggtcaccatggagtgctctccgagaacaggcctcgacttcaatgagatggtgttgctgcagatggaaaacaaagcttggctggtgcacaggcaatggttcctagacctgccgttaccatggctgcccggagcggatacacaagggtcaaattggatacagaaagaaacattggtcactttcaaaaatccccatgcgaagaaacaggatgttgttgttttaggatcccaagaaggggccatgcacacagcactcacaggggccacagaaatccaaatgtcatcaggaaatttactcttcacaggacatctcaagtgcaggctgagaatggacaagctacagctcaaaggaatgtcatactctatgtgcacaggaaagtttaaagttgtgaaggaaatagcagaaacacaacatggaacaatagttatcagagtgcaatatgaaggggacggctctccatgtaaaatcccttttgagataatggatttggaaaaaagacatgtcttaggtcgcctgatcacagtcaacccaattgtgacagaaaaagatagcccagtcaacatagaagcagaacctccattcggagacagctacatcatcataggagtagacccgggacaactgaagctcaactggtttaagaaaggaagttctatcggccaaatgtttgagacaacaatgaggggggcgaagagaatggccattttgggtgacacagcctgggattttggatccctgggaggagtgtttacatctataggaaaagctctccaccaagtctttggagcaatctatggagccgccttcagtggggtttcatggactatgaaaatcctcataggagtcattatcacatggataggaatgaattcacgcagcacctcactgtctgtgtcactagtattggtgggaattgtgacactgtatttgggagtcatggtgcaggcc

SEQ ID NO: 16 - protein, 495 aa, synthetic construct
MRCIGISNRDFVEGVSGGSWVDIVLEHGSCVTTMAKNKPTLDFELIKTEAKQPATLRKYCIEAKLTNTTTESRCPTQGEPSLNEEQDKRFVCKHSMVDRGWGSGCLLFGKGGIVTCAMFTCKKNMEGKVVQPENLEYTIVVTPHSGEEHAVGNDTGKHGKEIKVTPQSSIAEAELTGYGTVTMECSPRTGLDFNEMVLLQMENKAWLVHRQWFLDLPLPWLPGADTQGSNWIQKETLVTFKNPHAKKQDVVVLGSQEGAMHTALTGATEIQMSSGNLLFTGHLKCRLRMDKLQLKGMSYSMCTGKFKVVKEIAETQHGTIVIRVQYEGDGSPCKIPFEIMDLEKRHVLGRLITVNPIVTEKDSPVNIEAEPPFGDSYIIIGVDPGQLKLNWFKKGSSIGQMFETTMRGAKRMAILGDTAWDFGSLGGVFTSIGKALHQVFGAIYGAAFSGVSWTMKILIGVIITWIGMNSRSTSLSVSLVLVGIVTLYLGVMVQA

SEQ ID NO: 17 - protein, 166 aa, synthetic construct
FHLTTRNGEPHMIVSRQEKGKSLLFKTEDGVNMCTLMAMDLGELCEDTITYNCPLLRQNEPEDIDCWCNSTSTWVTYGTCTTTGAGRRSKRSVALVPHVGMGLETRTETWMSSEGAWKHAQRIETWILRHPGFTIMAAILAYTIGTTHFQRALIFILLTAVAPSMT

SEQ ID NO: 18 - protein, 13 aa, Dengue virus
DRGWGNGCGLFGK

SEQ ID NO: 19 - protein, 10 aa, Dengue virus
TGEHRREKRS